(19) United States
(12) Patent Application Publication
(10) Pub. No.: US 2005/0142578 A1
(43) Pub. Date: Jun. 30, 2005

(54) RNA interference mediated target discovery and target validation using short interfering nucleic acid (siNA)

(75) Inventors: Nassim Usman, Lafayette, CO (US); Barry Polisky, Boulder, CO (US); James Thompson, Lafayette, CO (US); Leonid Beigelman, Longmont, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirn Therapeutics, Inc., Boulder, CO (US)

(21) Appl. No.: 10/921,753

(22) Filed: Aug. 19, 2004

Related U.S. Application Data
(63) Continuation-in-part of application No. PCT/US03/04464, filed on Feb. 14, 2003.
(60) Provisional application No. 60/402,996, filed on Aug. 13, 2002.
(60) Provisional application No. 60/358,580, filed on Feb. 20, 2002.
(60) Provisional application No. 60/363,124, filed on Mar. 11, 2002.
(60) Provisional application No. 60/386,782, filed on Jun. 6, 2002.
(60) Provisional application No. 60/406,784, filed on Aug. 29, 2002.
(60) Provisional application No. 60/408,378, filed on Sep. 5, 2002.
(60) Provisional application No. 60/409,293, filed on Sep. 9, 2002.
(60) Provisional application No. 60/440,129, filed on Jan. 15, 2003.

(51) Int. Cl. C12Q 1/68; C07H 21/02

(52) U.S. Cl. 435/6; 536/23.1

(57) ABSTRACT

The present invention concerns methods and reagents useful in target discovery. Specifically, the invention relates to small nucleic acid molecules capable of mediating RNA interference (RNAi), such as short interfering nucleic acid (siNA) short interfering RNA (siRNA), double-stranded RNA (dsRNA), micro-RNA (miRNA), and short hairpin RNA (shRNA) molecules and methods of target discovery using siRNA.

This application is a continuation-in-part of International Patent Application No. PCT/US03/04464, filed Feb. 14, 2003, which claims the benefit of U.S. Provisional Application No. 60/402,996 filed Aug. 13, 2002, of U.S. Provisional Application No. 60/358,580 filed Feb. 20, 2002, of U.S. Provisional Application No. 60/363,124, filed Mar. 11, 2002, of U.S. Provisional Application No. 60/386,782, filed Jun. 6, 2002, of U.S. Provisional Application No. 60/406,784, filed Aug. 29, 2002, of U.S. Provisional Application No. 60/408,378, filed Sep. 5, 2002, of U.S. Provisional Application No. 60/409,293, filed Sep. 9, 2002, and of U.S. Provisional Application No. 60/440,129, filed Jan. 15, 2003. These applications are hereby incorporated by reference herein in their entireties, including the drawings.

### BACKGROUND OF THE INVENTION

The present invention concerns methods and reagents useful in target discovery and target validation, particularly genomic target discovery and validation. This invention also relates to a method for using small interfering nucleic acid (siNA) mediated RNA interference (RNAi) to identify accessible target sites in a cell to evaluate gene function, to validate a gene target for therapeutic intervention, and to identify and isolate nucleic acid molecules such as genes, involved in a biological process. Specifically, the invention relates to small nucleic acid molecules capable of mediating RNA interference (RNAi), such as short interfering nucleic acid (siNA) short interfering RNA (siRNA), double-stranded RNA (dsRNA), micro-RNA (miRNA), and short hairpin RNA (shRNA) molecules and methods of target discovery using siRNA.

The following is a discussion of relevant art pertaining to RNAi. The discussion is provided only for understanding of the invention that follows. The summary is not an admission that any of the work described below is prior art to the claimed invention.

RNA interference refers to the process of sequence-specific post-transcriptional gene silencing in animals mediated by short interfering RNAs (siRNAs) (Fire et al., 1998, Nature, 391, 806). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., 1999, Trends Genet., 15, 358). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Berstein et al., 2001, Nature, 409, 363). Short interfering RNAs derived from dicer activity are typically about 21 to about 23 nucleotides in length and comprise about 19 base pair duplexes (Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

RNAi has been studied in a variety of systems. Fire et al., 1998, Nature, 391, 806, were the first to observe RNAi in C. elegans. Wianny and Goetz, 1999, Nature Cell Biol., 2, 70, describe RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

Studies have shown that replacing the 3′-terminal nucleotide overhanging segments of a 21-mer siRNA duplex having two nucleotide 3′-overhangs with deoxyribonucleotides does not have an adverse effect on RNAi activity. Replacing up to four nucleotides on each end of the siRNA with deoxyribonucleotides has been reported to be well-tolerated, whereas complete substitution with deoxyribonucleotides results in no RNAi activity (Elbashir et al., 2001, EMBO J., 20, 6877). In addition, Elbashir et al., supra, also report that substitution of siRNA with 2′-O-methyl nucleotides completely abolishes RNAi activity. Li et al., International PCT Publication No. WO 00/44914, and Beach et al., International PCT Publication No. WO 01/68836 preliminarily suggest that siRNA may include modifications to either the phosphate-sugar backbone or the nucleoside to include at least one of a nitrogen or sulfur heteroatom, however, neither application postulates to what extent such modifications would be tolerated in siRNA molecules, nor provides any further guidance or examples of such modified siRNA. Kreutzer et al., Canadian Patent Application No. 2,359,180, also describe certain chemical modifications for use in dsRNA constructs in order to counteract activation of double-stranded RNA-dependent protein kinase PKR, specifically 2′-amino or 2′-O-methyl nucleotides, and nucleotides containing a 2′-O or 4′-C methylene bridge. However, Kreutzer et al. similarly fails to provide examples or guidance as to what extent these modifications would be tolerated in siRNA molecules.

Parrish et al., 2000, Molecular Cell, 6, 1977-1087, tested certain chemical modifications targeting the unc-22 gene in C. elegans using long (>25 nt) siRNA transcripts. The authors describe the introduction of thiophosphate residues into these siRNA transcripts by incorporating thiophosphate nucleotide analogs with T7 and T3 RNA polymerase and observed that RNAs with two phosphorothioate modified bases also had substantial decreases in effectiveness as RNAi. Further, Parrish et al. reported that phosphorothioate modification of more than two residues greatly destabilized the RNAs in vitro such that interference activities could not be assayed. Id. at 1081. The authors also tested certain modifications at the 2′-position of the nucleotide sugar in the long siRNA transcripts and found that substituting deoxynucleotides for ribonucleotides produced a substantial decrease in interference activity, especially in the case of Uridine to Thymidine and/or Cytidine to deoxy-Cytidine substitutions. Id. In addition, the authors tested certain base modifications, including substituting, in sense and antisense strands of the siRNA, 4-thiouracil, 5-bromouracil, 5-iodouracil, and 3-(aminoallyl)uracil for uracil, and inosine for guanosine. Whereas 4-thiouracil and 5-bromouracil substitution appeared to be tolerated, Parrish reported that inosine produced a substantial decrease in interference activity when incorporated in either strand. Parrish also reported that incorporation of 5-iodouracil and 3-(aminoallyl)uracil in the antisense strand resulted in a substantial decrease in RNAi activity as well.

The use of longer dsRNA has been described. For example, Beach et al., International PCT Publication No. WO 01/68836, describes specific methods for attenuating gene expression using endogenously-derived dsRNA. Tuschl et al., International PCT Publication No. WO 01/75164, describe a Drosophila in vitro RNAi system and the use of specific siRNA molecules for certain functional genomic and certain therapeutic applications; although Tuschl, 2001, Chem. Biochem., 2, 239-245, doubts that RNAi can be used to cure genetic diseases or viral infection due to the danger of activating interferon response. Li et al., International PCT Publication No. WO 00/44914, describe the use of specific dsRNAs for attenuating the expression of certain target genes. Zemicka-Goetz et al., International PCT Publication No. WO 01/36646, describe certain methods for inhibiting the expression of particular genes in mammalian cells using certain dsRNA molecules. Fire et al., International PCT Publication No. WO 99/32619, describe particular methods for introducing certain dsRNA molecules into cells for use in inhibiting gene expression. Plaetinck et al., International PCT Publication No. WO 00/01846, describe certain methods for identifying specific genes responsible for conferring a particular phenotype in a cell using specific dsRNA molecules. Mello et al., International PCT Publication No. WO 01/29058, describe the identification of specific genes involved in dsRNA-mediated RNAi. Deschamps Depaillette et al., International PCT Publication No. WO 99/07409, describe specific compositions consisting of particular dsRNA molecules combined with certain anti-viral agents. Waterhouse et al., International PCT Publication No. 99/53050, describe certain methods for decreasing the phenotypic expression of a nucleic acid in plant cells using certain dsRNAs. Driscoll et al., International PCT Publication No. WO 01/49844, describe specific DNA constructs for use in facilitating gene silencing in targeted organisms.

Others have reported on various RNAi and gene-silencing systems. For example, Parrish et al., 2000, Molecular Cell, 6, 1977-1087, describe specific chemically-modified siRNA constructs targeting the unc-22 gene of C. elegans. Grossniklaus, International PCT Publication No. WO 01/38551, describes certain methods for regulating polycomb gene expression in plants using certain dsRNAs. Churikov et al., International PCT Publication No. WO 01/42443, describe certain methods for modifying genetic characteristics of an organism using certain dsRNAs. Cogoni et al., International PCT Publication No. WO 01/53475, describe certain methods for isolating a Neurospora silencing gene and uses thereof. Reed et al., International PCT Publication No. WO 01/68836, describe certain methods for gene silencing in plants. Honer et al., International PCT Publication No. WO 01/70944, describe certain methods of drug screening using transgenic nematodes as Parkinson's Disease models using certain dsRNAs. Deak et al., International PCT Publication No. WO 01/72774, describe certain Drosophila-derived gene products that may be related to RNAi in Drosophila. Arndt et al., International PCT Publication No. WO 01/92513 describe certain methods for mediating gene suppression by using factors that enhance RNAi. Tuschl et al., International PCT Publication No. WO 02/44321, describe certain synthetic siRNA constructs. Pachuk et al., International PCT Publication No. WO 00/63364, and Satishchandran et al., International PCT Publication No. WO 01/04313, describe certain methods and compositions for inhibiting the function of certain polynucleotide sequences using certain dsRNAs. Echeverri et al., International PCT Publication No. WO 02/38805, describe certain C. elegans genes identified via RNAi. Kreutzer et al., International PCT Publications Nos. WO 02/055692, WO 02/055693, and EP 1144623 B1 describes certain methods for inhibiting gene expression using RNAi. Graham et al., International PCT Publications Nos. WO 99/49029 and WO 01/70949, and AU 4037501 describe certain vector expressed siRNA molecules. Fire et al., U.S. Pat. No. 6,506,559, describe certain methods for inhibiting gene expression in vitro using certain siRNA constructs that mediate RNAi.

Lofquist et al., 2002, U.S. Patent Application No. 20020094536, describes certain methods for making polynucleotide libraries, polynucleotide arrays, and cell libraries for high-throughput genomics analysis.

Reed et al., Australian Patent No. AU4037501; Waterhouse et al., International PCT Publication No. WO 99/53050; and Graham et al., International PCT Publication No. WO 01/70949, all describe certain RNAi methods and reagents for altering the phenotype of certain cells.

### SUMMARY OF THE INVENTION

The present invention features methods and reagents useful in genomic target discovery. This invention also features methods for using small interfering nucleic acid (siNA) mediated RNA interference (RNAi) to identify accessible target sites in a cell to evaluate gene function, to validate a gene target for therapeutic intervention, and/or to identify and isolate nucleic acid molecules, such as genes, involved in a biological process. Specifically, the invention features small nucleic acid molecules, such as short interfering nucleic acid (siNA) short interfering RNA (siRNA), double-stranded RNA (dsRNA), micro-RNA (miRNA), and short hairpin RNA (shRNA) molecules, capable of mediating RNA interference (RNAi) and methods of target discovery using such molecules.

In one embodiment, the invention features a method for identifying one or more nucleic acid molecules, such as gene(s), involved in a process in a biological system, the method comprising: (a) providing a library of siNA constructs to a biological system under conditions suitable for a process in the biological system to be altered; (b) identifying a siNA construct(s) present in the biological system in which a process has been altered; and (c) determining the nucleotide sequence of at least a portion of the siNA construct(s) in (b) to identify one or more nucleic acid molecules involved in a process in the biological system.

In another embodiment, the invention features a method for identifying a nucleic acid molecule capable of modulating a process in a biological system, the method comprising: (a) introducing a library of siNA constructs into a biological system under conditions suitable for modulating a process therein; and (b) determining the nucleotide sequence of at least a portion of a siNA construct from the biological system in which a process has been modulated to identify the nucleic acid molecule capable of modulating a process in the biological system.

In another embodiment, the invention features a method for identifying a siNA construct capable of modulating a process in a biological system, the method comprising: (a) introducing a library of siNA constructs into a biological system under conditions suitable for modulating a process therein; and b) identifying a siNA construct from the biological system in which a process has been modulated.

In one embodiment, the invention features a method for identifying a nucleic acid molecule or family of nucleic acid molecules, such as gene(s), involved in a process in a biological system, the method comprising: (a) providing a library of siNA constructs to a biological system under conditions suitable for a process in the biological system to be altered; (b) identifying a siNA construct or family of siNA constructs present in the biological system in which a process has been altered; and (c) determining the nucleotide sequence of at least a portion of the siNA construct or family of siNA constructs identified in (b) to identify the nucleic acid molecule or family of nucleic acid molecules involved in a process in the biological system.

In another embodiment, the invention features a method for identifying a nucleic acid molecule or family of nucleic acid molecules capable of modulating a process in a biological system, the method comprising: (a) introducing a library of siNA constructs into a biological system under conditions suitable for modulating a process therein; and (b) determining the nucleotide sequence of at least a portion of a siNA construct or family of siNA constructs from the biological system in which a process has been modulated to identify the nucleic acid molecule or family of nucleic acid molecules capable of modulating a process in the biological system.

In another embodiment, the invention features a method for identifying of a siNA construct or family of siNA constructs capable of modulating a process in a biological system, the method comprising: (a) introducing a library of siNA constructs into a biological system under conditions suitable for modulating a process therein; and b) identifying a siNA construct or family of siNA constructs from the biological system in which a process has been modulated.

In another embodiment, the invention features a method for identifying a gene that modulates a process in a biological system, comprising: (a) introducing a library of siNA constructs into a biological system under conditions suitable for modulating a process in the biological system, wherein each siNA comprises a randomized sense region and an antisense region having complementarity; (b) determining the nucleotide sequence of at least a portion of a siNA in the biological system in which a process has been modulated; and (c) identifying a gene that modulates a process in a biological system using the nucleotide sequence from (b).

In another embodiment, the invention features a method for identifying a gene that modulates a process in a biological system, comprising: (a) introducing a library of siNA constructs into a biological system under conditions suitable for modulating a process in the biological system, wherein each siNA comprises a randomized region; (b) determining the nucleotide sequence of at least a portion of a siNA in the biological system in which a process has been modulated; and (c) identifying a gene that modulates a process in a biological system using the nucleotide sequence from (b).

In another embodiment, the invention features a method for identifying a gene involved in a biological process, comprising: (a) introducing a library of siNA constructs into a biological system under conditions suitable for altering a process in the biological system, wherein each siNA comprises a randomized sense region and an antisense region having complementarity; (b) identifying a siNA in the biological system in which a biological process has been altered; and (c) determining the nucleotide sequence of at least a portion of the siNA from step (b) to identify a gene involved in the biological process.

In another embodiment, the invention features a method for identifying a gene involved in a biological process, comprising: (a) introducing a library of siNA constructs into a biological system under conditions suitable for altering a process in the biological system, wherein each siNA comprises a randomized region; (b) identifying a siNA in the biological system in which a biological process has been altered; and (c) determining the nucleotide sequence of at least a portion of the siNA from step (b) to identify a gene involved in the biological process.

In one embodiment, the invention features a method comprising: (a) providing a random siNA library to a biological system under conditions suitable for a siNA from the library to down-regulate the expression of a gene; (b) determining that gene expression has been down-regulated in the biological system (c) determining the nucleotide sequence of at least one portion of the siNA in the biological system of (b); and (d) identifying the gene which expression is down-regulated using the nucleotide sequence from (c). In one embodiment, a process contemplated by the invention comprises a biological process, such as processes including but not limited to, cell growth, proliferation, apoptosis, morphology, angiogenesis, differentiation, migration, viral multiplication, drug resistance, signal transduction, cell cycle regulation, morphogenesis, senesence, mitosis, meiosis, temperature sensitivity, chemical sensitivity, nerve cell growth, bacterial cell growth, plant cell growth, stress tolerance, biosynthesis of cellular factors or metabolites, viral resistance, bacterical resistence, or resistance to infection by a pathogen and others.

In one embodiment, the biological system of the invention comprises a cell, such as a eukaryotic cell or extract thereof, including but not limited to mammalian cells, such as human cells, plant cells, yeast cells, Drosophila cells, or C. elegans cells. In another embodiment, the biological system comprises a tissue or extract thereof. In another embodiment, the biological system comprises an organism or extract thereof.

One embodiment of the invention provides a short interfering RNA (siNA) molecule that down regulates expression of a gene by RNA interference, thus resulting in a phenotypic change that can be quantified. The siNA molecules can be used under conditions suitable to determine the sequence and/or identity of nucleic acid molecules (e.g. genes and gene transcipts) that are involved in or regulate biological processes described herein. The siNA molecule can comprise a double stranded RNA or a single stranded RNA. The double stranded or single stranded siNA can comprise a sense region and an antisense region. The antisense region can comprise sequence complementary to a nucleic acid molecule that regulates a biological process, and the sense region can comprise sequence complementary to the antisense region.

In one embodiment, a siNA molecule of the invention is a double stranded siNA molecule that mediates RNAi activity in a cell or reconstituted in vitro system, wherein the siNA molecule comprises a sense region and an antisense region in which the regions are self complementary and the antisense region has complementarity to a target nucleic acid sequence. In one embodiment, the double stranded siNA molecule of the invention comprises a single contigous nucleotide sequence having about 38 to about 58 nucleotides In another embodiment, the double stranded siNA molecule of the invention comprises separate sense and antisense regions, wherein each sense or antisense region independently comprises about 15 to about 30 nucleotides.

In one embodiment, a siNA molecule of the invention is a single stranded siNA molecule that mediates RNAi activity in a cell or reconstituted in vitro system, wherein the siNA molecule comprises a single stranded polynucleotide having complementarity to a target nucleic acid sequence. In one embodiment, the single stranded siNA molecule of the invention comprises about 15 to about 30 nucleotides.

In one embodiment, a siNA library of the invention comprises a library of randomized siNA sequences. In another embodiment, a library of random siNA molecules of the invention comprises sequence complexity such that any member of the library has an antisense sequence that is complementary to any nucleic acid molecule in a biological system that regulates a process therein. The sequence can be partially random or completely random (see, for example, Keck et al., International PCT Publication No. WO 99/32618). The siNA library can be of fixed or variable sequence length. The degree of complexity of the library can therefore depend on the number of nucleotides in a predetermined siNA construct. For example, a siNA comprising a sense and antisense strand each having N nucleotides can have a library complexity of 4^{N}, where N is the number of nucleotides in the antisense strand (4 relates to the number of naturally occurring nucleotides, A, G, C or U, present in a target nucleic acid sequence in a biological system of the invention). In a non-limiting example, a siNA comprising a sense and antisense strand each having 21 nucleotides can have a library complexity of 4²¹, such that every possible combination of complementary nucleotides is included in the library. In yet another embodiment, the complexity of the library is modulated under conditions suitable to identify a target nucleic acid molecule of the invention.

A random library of siNA constructs can comprise siNA constructs encoded by an expression vector in a manner that allows expression of said siNA constructs. In one embodiment, the expression vector comprises a transcription initiation region, a transcription termination region, and a gene encoding at least one siNA. The gene can be operably linked to the initiation region and the termination region, in a manner which allows expression and/or delivery of the siNA. In another embodiment, the expression vector can comprises a transcription initiation region, a transcription termination region, an open reading frame and a gene encoding at least one siNA, wherein the gene is operably linked to the 3′-end of the open reading frame. The gene can be operably linked to the initiation region, the open reading frame and the termination region in a manner which allows expression and/or delivery of the siNA. In another embodiment, the expression vector comprises a transcription initiation region, a transcription termination region, an intron, and a gene encoding at least one siNA. The gene can be operably linked to the initiation region, the intron, and the termination region in a manner which allows expression and/or delivery of the siNA. In yet another embodiment, the expression vector comprises a transcription initiation region, a transcription termination region, an intron, an open reading frame, and a gene encoding at least one siNA, wherein the gene is operably linked to the 3′-end of the open reading frame. The gene can be operably linked to the initiation region, the intron, the open reading frame and the termination region in a manner which allows expression and/or delivery of the siNA.

The expression vector can be derived from, for example, a retrovirus, an adenovirus, an adeno-associated virus, an alphavirus or a bacterial plasmid as well as other known vectors. The expression vector can be operably linked to a RNA polymerase II promoter element or a RNA polymerase III promoter element. The RNA polymerase III promoter can be derived from, for example, a transfer RNA gene, a U6 small nuclear RNA gene, or a TRZ RNA gene. The siNA transcript can comprise a sequence at its 5′-end homologous to the terminal 27 nucleotides encoded by the U6 small nuclear RNA gene. The library of siNA constructs can be a multimer random library. The multimer random library can comprise at least one siNA.

The siNA of the instant invention can be chemically synthesized, expressed from a vector, or enzymatically synthesized.

In one embodiment, a siNA molecule of the invention is chemically modified and is used to validate a target identified by a method of the invention. Such chemically modified siNA constructs can also be used to identify suitable pharmaceutical development candidates having optimal activity in modulating the expression of a target identified by a method of the invention or by other methods. Such constructs can be used in high throughput screening approaches to optimization of target sites and/or optimization of pharmaceutical leads. Non-limiting examples of the design and synthesis of chemically modified siNA constructs useful in this invention are described in Beigelman et al., U.S. Ser. No. 60/358,580, incorporated by reference herein in its entirety including the drawings.

In one embodiment, the sense region of a siNA molecule of the invention comprises a 3′-terminal overhang. In another embodiment, the antisense region of a siNA molecule comprises a 3′-terminal overhang. In another embodiment, both the sense and the antisense regions comprise a 3′-terminal overhang. In one embodiment, the siNA comprises a 3′-terminal overhang of about 1 to about 3 nucleotides in the sense region, or the antisense region, or both the sense and antisense regions of the siNA. In one embodiment, the 3′-terminal overhangs each comprise about 2 nucleotides. In one embodiment, the antisense region of the 3′-terminal nucleotide overhang can be complementary to the target RNA. The siNA is of a length sufficient to mediate RNAi. The siNA can comprise a sense and antisense region, wherein each region has a length of about 15 to about 30 nucleotides.

In one embodiment, the invention provides an expression vector comprising a nucleic acid sequence encoding at least one siNA molecule of the invention in a manner that allows expression of the nucleic acid molecule. The expression vector can be in a mammalian cell, such as a human cell. The siNA molecule can comprise a sense region and an antisense region. The antisense region can comprise sequence complementary to any nucleic acid molecule in a biological system that regulates a process therein and the sense region can comprise sequence complementary to the antisense region. The siNA molecule can comprise two distinct strands having complementary sense and antisense regions or can comprise a single strand having complementary sense and antisense regions.

Therefore, this invention relates to compounds, compositions, and methods useful for target discovery through modulation of gene expression, for example, genes associated with biological processes, by RNA interference (RNAi) using short interfering nucleic acid (siNA). In particular, the instant invention features siNA molecules and methods to modulate the expression of genes associated with biological processes to determine the function of a known sequence, or to determine the sequence of a nucleic acid molecule associated with a particular phenotype.

In one embodiment, the invention features one or more siNA molecules and methods that independently or in combination modulate the expression of gene(s) that are associated with a particular phenotype or that regulate a particular process in a biological system. In another embodiment, an observed change in phenotype that is associated with modulation of gene expression via a siNA construct of the invention is used to determine the function and/or sequence of a particular nucleic acid molecule present in a biological system.

In one embodiment, the invention features a siNA molecule having RNAi activity against a cellular RNA, wherein the siNA molecule comprises a sequence (e.g. antisense sequence) complementary to the target RNA.

In one embodiment, nucleic acid molecules (e.g., siNA) of the invention that act as mediators of the RNA interference gene silencing response are double stranded RNA molecules. In another embodiment, the siNA molecules of the invention consist of duplexes containing about 19 base pairs between oligonucleotides comprising about 15 to about 30 nucleotides (e.g., about 15, 16 17, 18, 19, 20, 21, 22, 23, 24, 25, 26, 27, 28, 29, or 30). In yet another embodiment, siNA molecules of the invention comprise duplexes with overhanging ends of 1-3 (e.g., 1, 2, or 3) nucleotides, for example, 21 nucleotide duplexes with 19 base pairs and 2 nucleotide 3′-overhangs. These nucleotide overhangs in the antisense strand are optionally complementary to the target sequence.

In one embodiment, nucleic acid molecules (e.g., siNA) of the invention that act as mediators of the RNA interference gene silencing response are single stranded RNA molecules. In another embodiment, single stranded nucleic acid molecules (e.g., siNA) of the invention consist of sequence comprising about 15 to about 30 nucleotides (e.g., about 15, 16, 17, 18, 19, 20, 21, 22, 23, 24, 25, 26, 27, 28, 29, or 30).

In one embodiment, nucleic acid molecules (e.g., siNA) of the invention that act as mediators of the RNA interference gene silencing response are single stranded RNA molecules having a stem-loop structure. The stem can comprise self complementary sense and antisense regions of the siNA, wherein for example each sense and antisense region independently comprises about 15 to about 30 nucleotides, (e.g., about 15, 16, 17, 18, 19, 20, 21, 22, 23, 24, 25, 26, 27, 28, 29 or 30 nucleotides). The loop portion of the siNA can comprise about 2 to about 10 or more nucleotides (e.g., about 2, 3, 4, 5, 6, 7, 8, 9, 10 or more nucleotides). In addition, the stem portion of the siNA stem/loop construct can also comprise a 3′-overhang having about 1 to about 4 or more nucleotides, (e.g., about 1, 2, 3, 4, or more nucleotides).

In another embodiment, a linear hairpin siNA molecule of the invention contains a stem loop motif, for example, wherein the loop portion of the siNA molecule is biodegradable. For example, a linear hairpin siNA molecule of the invention is designed such that degradation of the loop portion of the siNA molecule in vivo can generate a double stranded siNA molecule with 3′-overhangs (such as 3′-overhangs comprising about 2 nucleotides) or alternately no overhangs.

In another embodiment, a siNA molecule of the invention comprises a circular nucleic acid molecule, for example wherein the siNA is about 38 to about 70 (e.g., about 38, 40, 45, 50, 55, 60, 65, or 70) nucleotides in length having about 15 to about 30 (e.g., about 15, 16, 17, 18, 19, 20, 21, 22, 23, 24, 25, 26, 27, 28, 29, or 30) base pairs. For example, an exemplary siNA molecule of the invention comprises a circular oligonucleotide having about 42 to about 50 (e.g., about 42, 43, 44, 45, 46, 47, 48, 49, or 50) nucleotides, wherein the circular oligonucleotide forms a dumbbell shaped structure having 19 base pairs and 2 loops.

In another embodiment, a circular siNA molecule of the invention contains two loop motifs, wherein one or both loop portions of the siNA molecule is biodegradable. For example, a circular siNA molecule of the invention is designed such that degradation of the loop portions of the siNA molecule in vivo can generate a double stranded siNA molecule with 3′-overhangs, such as 3′-overhangs comprising about 2 nucleotides.

The siNA molecules of the invention can be designed to inhibit gene expression through RNAi targeting of a variety of RNA molecules. In one embodiment, the siNA molecules of the invention are used to target various RNAs corresponding to a target gene. Non-limiting examples of such RNAs include messenger RNA (mRNA), alternate RNA splice variants of target gene(s), post-transcriptionally modified RNA of target gene(s), pre-mRNA of target gene(s), and/or RNA templates. If alternate splicing produces a family of transcipts that are distinguished by usage of appropriate exons, the instant invention can be used to inhibit gene expression through the appropriate exons to specifically inhibit or to distinguish among the functions of gene family members. For example, a protein that contains an alternatively spliced transmembrane domain can be expressed in both membrane bound and secreted forms. Use of the invention to target the exon containing the transmembrane domain can be used to determine the functional consequences of pharmaceutical targeting of membrane bound as opposed to the secreted form of the protein. Non-limiting examples of applications of the invention relating to targeting these RNA molecules include therapeutic pharmaceutical applications, pharmaceutical discovery applications, molecular diagnostic and gene function applications, and gene mapping, for example using single nucleotide polymorphism mapping with siNA molecules of the invention. Such applications can be implemented using known gene sequences or from partial sequences available from an expressed sequence tag (EST).

In another embodiment, the siNA molecules of the invention are used to target conserved sequences corresponding to a gene family or gene families. As such, siNA molecules targeting multiple gene targets can provide increased therapeutic effect. In addition, siNA can be used to characterize pathways of gene function in a variety of applications. For example, the present invention can be used to inhibit the activity of target gene(s) in a pathway to determine the function of uncharacterized gene(s) in gene function analysis, mRNA function analysis, or translational analysis. The invention can be used to determine potential target gene pathways involved in various diseases and conditions toward pharmaceutical development. The invention can be used to understand pathways of gene expression involved in development, such as prenatal development, postnatal development and/or aging.

In one embodiment, a siNA molecule of the invention has RNAi activity that modulates expression of RNA encoded by a target gene. Because related genes typically share some degree of sequence homology with each other, siNA molecules can be designed to target a class of target genes or alternately specific target genes by selecting sequences that are either shared amongst different target genes or that are alternately unique for a specific target gene. Therefore, in one embodiment, the siNA molecule can be designed to target conserved regions of RNA sequence having homology between several target genes so as to target several genes or gene families (e.g., splice variants, mutant genes etc.) with one siNA molecule. In another embodiment, the siNA molecule can be designed to target a sequence that is unique to a specific RNA sequence due to the high degree of specificity that the siNA molecule requires to mediate RNAi activity. In one embodiment, the invention features a method comprising: (a) generating a randomized library of siNA constructs having a predetermined complexity, such as of 4^{N}, where N represents the number of base paired nucleotides in each of the siNA construct strands (e.g. for a siNA construct having 21 nucleotide sense and antisense strands with 19 base pairs, the complexity would be 4¹⁹); and (b) assaying the siNA constructs of (a) above, under conditions suitable to determine RNAi target sites within the target RNA sequence. In another embodiment, the siNA molecules of (a) have strands of a fixed length, for example about 23 nucleotides in length. In yet another embodiment, the siNA molecules of (a) are of differing length, for example having strands of about 15 to about 30 (e.g., about 15, 16, 17, 18, 19, 20, 21, 22, 23, 24, 25, 26, 27, 28, 29, or 30) nucleotides in length. In yet another embodiment, the assay can comprise a reconstituted in vitro siNA assay as described herein. In another embodiment, the assay can comprise a cell culture system in which target RNA is expressed. In another embodiment; fragments of RNA are analyzed for detectable levels of cleavage, for example by gel electrophoresis, northern blot analysis, or RNAse protection assays, to determine the most suitable target site(s) within the target RNA sequence. In another embodiment, the target RNA sequence can be obtained as is known in the art, for example, by cloning and/or transcription for in vitro systems, and by cellular expression in in vivo systems.

In another embodiment, the invention features a method comprising: (a) analyzing the sequence of a RNA target encoded by a gene; (b) synthesizing one or more siNA molecules having sequence complementary to one or more regions of the RNA of (a); and (c) assaying the siNA molecules of (b) under conditions suitable to determine RNAi targets within the target RNA sequence. In another embodiment, the siNA molecules of (b) have strands of a fixed length, for example about 23 nucleotides in length. In yet another embodiment, the siNA molecules of (b) are of differing length, for example having strands of about 15 to about 30 (e.g., about 15, 16, 17, 18, 19, 20, 21, 22, 23, 24, 25, 26, 27, 28, 29 or 30) nucleotides in length. In yet another embodiment, the assay can comprise a reconstituted in vitro siNA assay as described herein. In another embodiment, the assay can comprise a cell culture system in which target RNA is expressed. Fragments of RNA are analyzed for detectable levels of cleavage, for example by gel electrophoresis, northern blot analysis, or RNAse protection assays, to determine the most suitable target site(s) within the target RNA sequence. The target RNA sequence can be obtained as is known in the art, for example, by cloning and/or transcription for in vitro systems, and by expression in in vivo systems.

By “target site” is meant a sequence within a target RNA that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection known to those skilled in the art.

In one embodiment, the invention features a composition comprising a siNA molecule of the invention, which can be chemically modified, in a pharmaceutically acceptable carrier or diluent. In another embodiment, the invention features a composition comprising one or more siNA molecules of the invention, which can be chemically modified, targeting one or more genes in a pharmaceutically acceptable carrier or diluent. In another embodiment, the invention features a method for treating or preventing a disease or condition in a subject, comprising administering to the subject a composition of the invention under conditions suitable for the treatment or prevention of the disease or condition in the subject, alone or in conjunction with one or more other therapeutic compounds. In yet another embodiment, the invention features a method for reducing or preventing tissue rejection in a subject comprising administering to the subject a composition of the invention under conditions suitable for the reduction or prevention of tissue rejection in the subject.

In another embodiment, the invention features a method for validating a gene target, comprising: (a) synthesizing a siNA molecule of the invention, which can be chemically modified, wherein one of the siNA strands includes a sequence complementary to RNA of a target gene; (b) introducing the siNA molecule into a biological system under conditions suitable for modulating expression of the target gene in the biological system; and (c) determining the function of the gene by assaying for any phenotypic change in biological system.

In another embodiment, the invention features a method for validating a gene target, comprising: (a) synthesizing a siNA molecule of the invention, which can be chemically modified, wherein the siNA comprises a sequence complementary to RNA of a target gene; (b) introducing the siNA molecule into a biological system under conditions suitable for modulating expression of the target gene in the biological system; and (c) determining the function of the gene by assaying for any phenotypic change in the biological system.

In one embodiment of the described methods for validating a gene target, the siNA molecule is single stranded. In another embodiment of the method, the siNA molecule is double stranded. The siNA molecule used in the inventive method can be chemically modified, for example, as described herein. The biological system of the inventive method can be, for example, a cell, tissue, or organism, or extract thereof.

In one embodiment, the invention features a kit containing a siNA molecule of the invention, which can be chemically modified, that can be used to modulate the expression of a target gene in a biological system. In another embodiment, the invention features a kit containing more than one siNA molecule of the invention, which can be chemically modified, that can be used to modulate the expression of more than one target gene in a biological system.

In one embodiment, the invention features a cell containing one or more siNA molecules of the invention, which can be chemically modified. In another embodiment, the cell containing a siNA molecule of the invention is a mammalian cell. In yet another embodiment, the cell containing a siNA molecule of the invention is a human cell.

The present invention can be used alone or as a component of a kit having at least one of the reagents necessary to carry out the in vitro or in vivo introduction of RNA to test samples and/or subjects. For example, preferred components of the kit include the siNA and a vehicle that promotes introduction of the siNA. Such a kit can also include instructions to allow a user of the kit to practice the invention.

By “biological system” is meant, material, in a purified or unpurified form, from biological sources, including but not limited to human, animal, plant, insect, bacterial, viral or other sources, wherein the system comprises the components required for RNAi acitivity. The term “biological system” can include a cell, tissue, or organism, or extract thereof. The term biological system also includes reconstituted RNAi systems that can be used in an in vitro setting.

By “phenotypic change” is meant any detectable change to a cell that occurs in response to contact or treatment with a nucleic acid molecule of the invention (e.g., siNA). Such detectable changes include, but are not limited to, changes in shape, size, proliferation, motility, protein expression or RNA expression or other physical or chemical changes as can be assayed by methods known in the art. The detectable change can also include expression of reporter genes/molecules such as Green Florescent Protein (GFP) or various tags that are used to identify an expressed protein or any other cellular component that can be assayed.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference “RNAi” or gene silencing in a sequence-specific manner; see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Non limiting examples of siNA molecules of the invention are shown in FIG. 2 herein. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see for example Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “family of siNA constructs” is meant a group of more than one siNA constructs that share at least one common characteristic, such as sequence homology, target specificity, mode of action, secondary structure, or the ability to modulate a process or more than one process in a biological system.

By “family of nucleic acid molecules” is meant a group of more than one nucleic acid molecules that share at least one common characteristic, such as sequence homology, target specificity, mode of action, secondary structure, or the ability to modulate a process or more than one process in a biological system.

By “modulate” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit,” but the use of the word “modulate” is not limited to this definition, e.g., “modulate” can also refer to activation by either a direct or indirect mechanism of action.

By “inhibit” it is meant that the activity of a gene expression product or level of RNAs or equivalent RNAs encoding one or more gene products is reduced below that observed in the absence of the nucleic acid molecule of the invention. In one embodiment, inhibition with a siNA molecule preferably is below that level observed in the presence of an inactive or attenuated molecule that is unable to mediate an RNAi response. In another embodiment, inhibition of gene expression with the siNA molecule of the instant invention is greater in the presence of the siNA molecule than in its absence.

By “gene” or “target gene” is meant a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

By “highly conserved sequence region” is meant a nucleotide sequence of one or more regions in a target gene that does not vary significantly from one generation to the other or from one biological system to the other.

By “complementarity” or “complementary” is meant that a nucleic acid can form hydrogen bond(s) with another nucleic acid sequence by either traditional Watson-Crick or other non-traditional types of interaction. In reference to the nucleic molecules of the present invention, the binding free energy for a nucleic acid molecule with its complementary sequence is sufficient to allow the relevant function of the nucleic acid to proceed, e.g., RNAi activity. For example, the degree of complementarity between the sense and antisense strand of the siNA construct can be the same or different from the degree of complementarity between the antisense strand of the siNA and the target RNA sequence. Complementarity to the target sequence of less than 100% in the antisense strand of the siNA duplex, including point mutations, is reported not to be tolerated when these changes are located between the 3′-end and the middle of the antisense siNA (completely abolishes siNA activity), whereas mutations near the 5′-end of the antisense siNA strand can exhibit a small degree of RNAi activity (Elbashir et al., 2001, The EMBO Journal, 20, 6877-6888). Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

In one embodiment of the present invention, each sequence of a siNA molecule of the invention is independently about 18 to about 23 nucleotides in length, in specific embodiments about 18, 19, 20, 21, 22, or 23 nucleotides in length. In one embodiment of the present invention, each sequence of a siNA molecule of the invention is independently about 18 to about 28 nucleotides in length, in specific embodiments about 18, 19, 20, 21, 22, 23, 24, 25, 26, 27, or 28 nucleotides in length. In one embodiment of the present invention, each sequence of a siNA molecule of the invention is independently about 15 to about 30 nucleotides in length, in specific embodiments about 15, 16, 17, 18, 19, 20, 21, 22, 23, 24, 25, 26, 27, 28, 29, or 30 nucleotides in length. In one embodiment of the present invention, each sequence of a siNA molecule of the invention is independently about 15 to about 40 nucleotides in length, in specific embodiments about 15, 16, 17, 18, 19, 20, 21, 22, 23, 24, 25, 26, 27, 28, 29, 30, 31, 32, 33, 34, 35, 36, 37, 38, 39, or 40 nucleotides in length. In another embodiment, the siNA duplexes of the invention independently comprise about 17 to about 23 (e.g., about 17, 18, 19, 20, 21, 22, or 23) base pairs. In yet another embodiment, siNA molecules of the invention comprising hairpin or circular structures are about 35 to about 55 (e.g., about 35, 40, 45, 50, or 55) nucleotides in length, or about 38 to about 44 (e.g., about 38, 39, 40, 41, 42, 43, or 44) nucleotides in length and comprise about 15 to about 25 (e.g., about 15, 16, 17, 18, 19, 20, 21, 22, 23, 24, or 25) base pairs.

As used herein “cell” is used in its usual biological sense, and does not refer to an entire multicellular organism, e.g., specifically does not refer to a human. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell.

The siNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers.

In another aspect, the invention provides mammalian cells containing one or more siNA molecules of this invention. The one or more siNA molecules can independently be targeted to the same or different sites.

By “RNA” is meant a molecule comprising at least one ribonucleotide residue. By “ribonucleotide” is meant a nucleotide with a hydroxyl group at the 2′ position of a β-D-ribo-furanose moiety. The terms include double stranded RNA, single stranded RNA, isolated RNA, such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

In one embodiment, the invention features an expression vector comprising a nucleic acid sequence encoding at least one siNA molecule of the invention, in a manner which allows expression of the siNA molecule. For example, the vector can contain sequence(s) encoding both strands of a siNA molecule comprising a duplex. The vector can also contain sequence(s) encoding a single nucleic acid molecule that is self complementary and thus forms a siNA molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725.

In another embodiment, the invention features a mammalian cell, for example, a human cell, including an expression vector of the invention.

In one embodiment, an expression vector of the invention comprises a nucleic acid sequence encoding two or more siNA molecules, which can be the same or different.

In another aspect of the invention, siNA molecules that interact with target RNA molecules and down-regulate gene encoding target RNA molecules are expressed from transcription units inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. The recombinant vectors capable of expressing the siNA molecules can be delivered as described herein, and persist in target cells. Alternatively, viral vectors can be used that provide for transient expression of siNA molecules. Such vectors can be repeatedly administered as necessary. Once expressed, the siNA molecules bind and down-regulate gene function or expression via RNA interference (RNAi). Delivery of siNA expressing vectors can be systemic, such as by intravenous or intramuscular administration, by administration to target cells ex-planted from a subject followed by reintroduction into the subject, or by any other means that would allow for introduction into the desired target cell.

By “vectors” is meant any nucleic acid- and/or viral-based technique used to deliver a desired nucleic acid. Other features and advantages of the invention will be apparent from the following description of the preferred embodiments thereof, and from the claims.

### BRIEF DESCRIPTION OF THE DRAWINGS

FIG. 1 is a schematic depicting a non-limiting proposed mechanistic representation of target RNA degradation involved in RNAi. Double stranded RNA (dsRNA), generated from, for example, viral, transposon, or other exogenous RNA, activates the DICER enzyme which in turn generates siNA duplexes having terminal phosphate groups (P). An active siNA complex forms which recognizes a target RNA, resulting in degradation of the target RNA by the RISC endonuclease complex or in the synthesis of additional RNA by RNA dependent RNA polymerase (RdRP), which can activate DICER and result in additional siNA molecules, thereby amplifying the RNAi response.

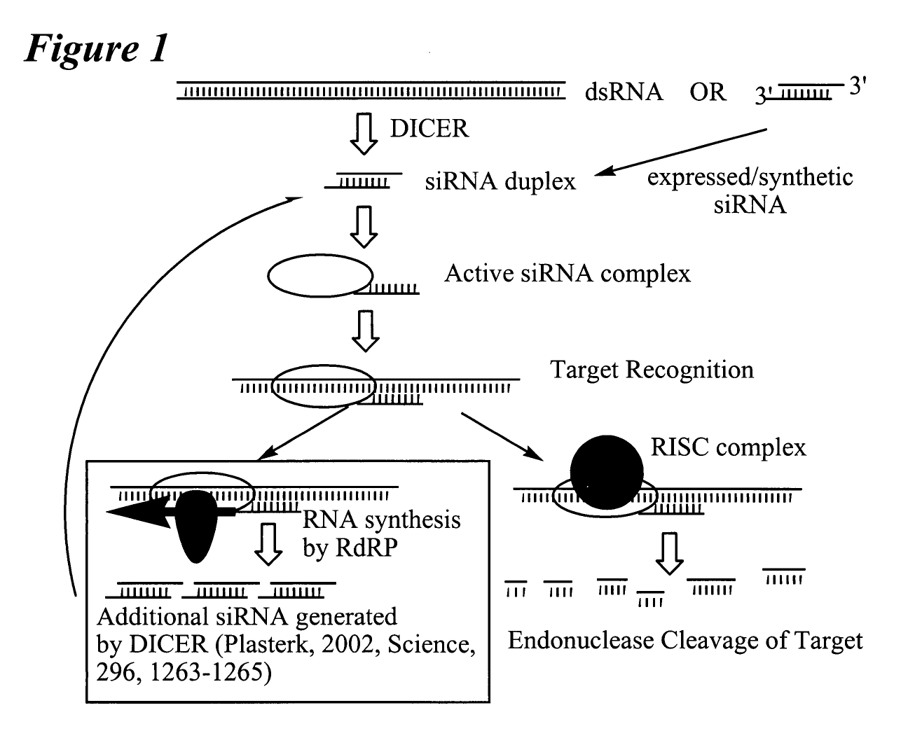

FIG. 2 shows non-limiting examples of different siNA constructs of the invention. The examples shown (constructs 1, 2, and 3) have 19 representative base pairs. However, different embodiments of the invention include any number of base pairs described herein. Bracketed regions represent nucleotide overhangs, for example comprising about 0, 1, 2, 3, or 4 nucleotides in length, preferably about 2 nucleotides. Constructs 1 and 2 can be used independently to induce RNAi activity. Construct 2 can comprise a polynucleotide or non-nucleotide linker, which can optionally be designed as a biodegradable linker. In one embodiment, the loop structure shown in construct 2 can comprise a biodegradable linker that results in the formation of construct 1 in vivo and/or in vitro. In another example, construct 3 can be used to generate construct 2 under the same principle wherein a linker is used to generate the active siNA construct 2 in vivo and/or in vitro, which can optionally utilize another biodegradable linker to generate the active siNA construct 1 in vivo and/or in vitro. As such, the stability and/or activity of the siNA constructs can be modulated based on the design of the siNA construct for use in vivo or in vitro and/or in vitro.

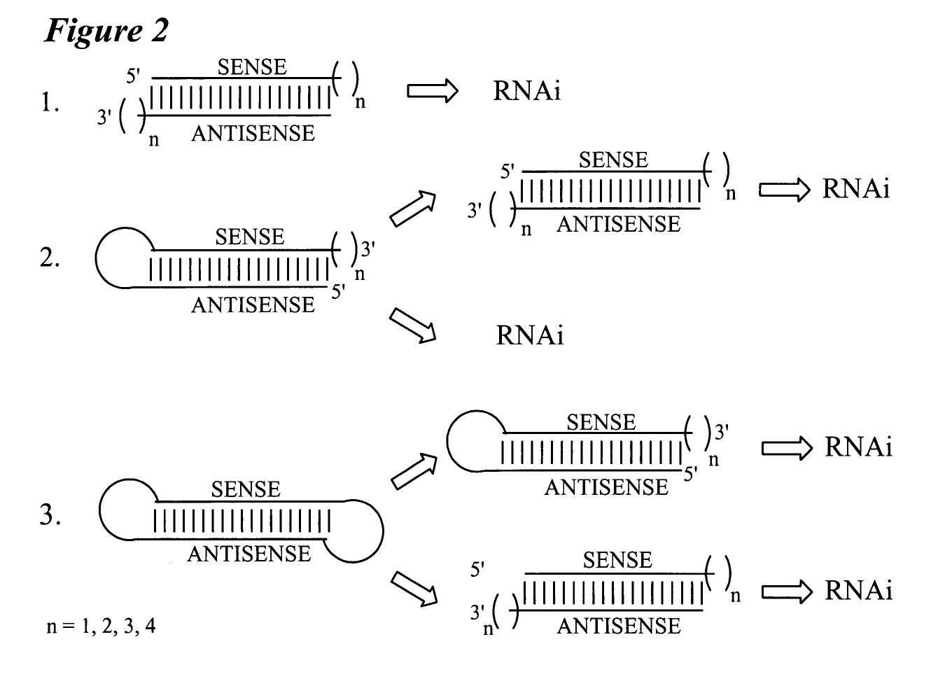

FIG. 3 shows a non-limiting example of siNA constructs used to generate a completely random library of the instant invention. The examples shown are double stranded siNA constructs, however, single stranded siNA constructs comprising hairpin loops can likewise be used to generate libraries of the invention; such as for use in target discovery.

FIG. 4 shows a non-limiting example of diagrammatic schemes representative of the process used for Target Discovery in the instant invention. An siNA library is prepared in which all 4 standard nucleotides are incorporated in a random fashion in the antisense region of the siNA to produce a pool of all possible siNA molecules with complementary sense regions for a given sequence length. This pool is cloned into an appropriate vector to produce the Random Library expression vector and retroviral vector particles are produced from this pool. The resulting Random siNA Library retroviral expression vector pool is then used to transduce a cell type of interest. Cells exhibiting the desired phenotype are then separated from the rest of the population using a number of possible selection strategies. Genes that are critical for expression of the selected phenotype can then be identified by sequencing the siNA contained in the selected population.

FIG. 5 is a schematic showing a non-limiting example of a general approach to target discovery in plant cells using siNA constructs of the invention.

FIG. 6 shows a non-limiting example of a general approach to target discovery and determination of accessible target sites using siNA constructs of the invention.

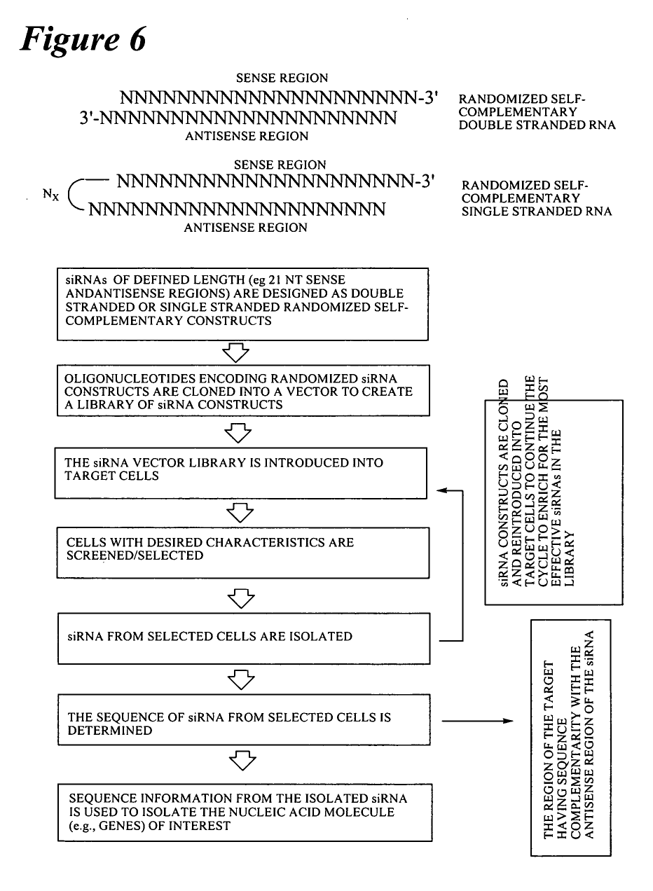

FIG. 7 is a diagrammatic representation of multimer and monomer random library constructs. In the case of the monomer random library transcript, a single hairpin or single double stranded siNA construct is transcribed from the transcript. The double stranded siNA construct is generated, for example from a restriction enzyme cleavage site, or alternately by a separate transcript. In the case of the multimer random library transcript, several hairpin or several double stranded siNA constructs are encoded in the same trascript. The double stranded siNA construct is generated, for example from a restriction enzyme cleavage sites, or alternately by a separate transcript. Non-limiting examples of siNA hairpin expression systems are described in, for example, Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi:10.1038/nm725.

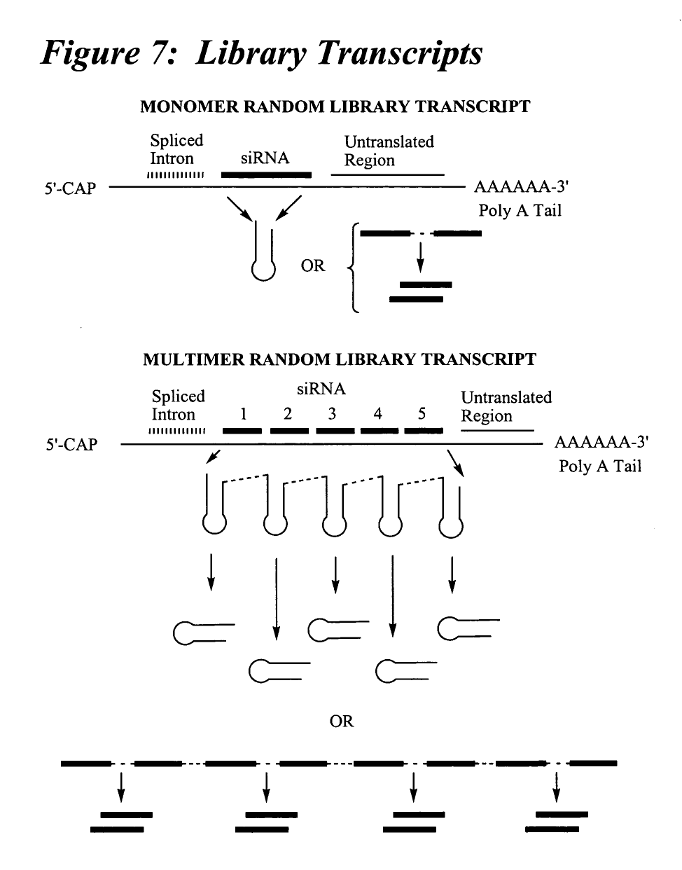

FIG. 8 is a diagrammatic representation of a scheme utilized in generating an expression cassette to generate a random siNA hairpin library of the invention. (A) A DNA oligomer is synthesized with a 5′-restriction site (R1) sequence followed by a randomized region, for example 19, 20, 21, or 22 nucleotides (N) in length, which is followed by a stem-loop sequence of defined sequence (X). The random region is generated as is known in the art, for example by mixing combinations of phosphoramidites during solid phase oligonucleotide synthesis. (B) The synthetic construct is then extended by DNA polymerase to generate a randomized hairpin structure having self complementary sequence that will result in a siNA transcript having randomized self complementary sense and antisense regions. (C) The construct is heated (for example to about 95° C.) to linearize the sequence, thus allowing extension of a complementary second DNA strand using a primer to the 3′-restriction sequence of the first strand. The double stranded DNA is then inserted into an appropriate vector for expression in cells. The construct can be designed such that a 3′-overhang results from the transcription, for example by engineering restriction sites and/or utilizing a poly-U termination region as described in Paul et al., 2002, Nature Biotechnology, 29, 505-508.

FIG. 9 is a diagrammatic representation of a scheme utilized in generating an expression cassette to generate a random double stranded siNA library of the invention. (A) A DNA oligomer is synthesized with a 5′-restriction (R1) site sequence followed by a randomized region, for example 19, 20, 21, or 22 nucleotides (N) in length, which is followed by a 3′-restriction site (R2) which is adjacent to a stem-loop sequence of defined sequence (X). The random region is generated as is known in the art, for example by mixing combinations of phosphoramidites during solid phase oligonucleotide synthesis. (B) The synthetic construct is then extended by DNA polymerase to generate a randomized hairpin structure having self complementary sequence. (C) The construct is processed by restriction enzymes specific to R1 and R2 to generate a double stranded DNA which is then inserted into an appropriate vector for expression in cells. The transcription cassette is designed such that a U6 promoter region flanks each side of the dsDNA which generates the separate sense and antisense strands of the siNA library components. Poly T termination sequences can be added to the constructs to generate U overhangs in the resulting transcript.

### DETAILED DESCRIPTION OF THE INVENTION

Mechanism of Action of Nucleic Acid Molecules of the Invention

RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as Dicer. Dicer is involved in the processing of the dsRNA into short pieces of dsRNA known as short interfering RNAs (siRNA) (Berstein et al., 2001, Nature, 409, 363). Short interfering RNAs derived from Dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188). In addition, RNA interference can also involve small RNA (e.g., micro-RNA or miRNA) mediated gene silencing, presumably though cellular mechanisms that regulate chromatin structure and thereby prevent transcription of target gene sequences (see for example Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). As such, siNA molecules of the invention can be used to mediate gene silencing via interaction with RNA transcripts or alternately by interaction with particular gene sequences, wherein such interaction results in gene silencing either at the transcriptional level or post-transcriptional level.

Short interfering RNA mediated RNAi has been studied in a variety of systems. Fire et al., 1998, Nature, 391, 806, were the first to observe RNAi in C. elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs can occur in vivo.

Screening Methods:

Applicant has developed an efficient and rapid method for screening libraries of siNA molecules capable of performing a desired function in a cell. The invention also features the use of a siNA library to modulate certain attributes or processes in a biological system, such as a mammalian cell, and to identify and isolate (a) siNA molecules from the library involved in modulating the cellular process or attribute of interest; and b) modulators of the desired cellular process or attribute using the sequence of the siNA.

More specifically, the method of the instant invention involves designing and constructing a siNA library, where the siNA includes randomized sequence (e.g. randomized antisense strand with a complementary sense strand). This library of siNA molecules with randomized self complementary sequence are used to modulate certain processes or attributes in a biological system. The method described herein involves simultaneous screening of a library or pool of siNA molecules with various substitutions at one or more positions and selecting for siNA with desired function or characteristics or attributes. This invention also features a method for constructing and selecting for siNA molecules for their ability to cleave a given target nucleic acid molecule or an unknown target nucleic acid molecule (e.g., RNA), and to inhibit the biological function of that target molecule or any protein encoded by it.

It is not necessary to know either the sequence or the structure of the target nucleic acid molecule in order to select for siNA molecules capable of mediating RNAi based cleavage of the target in this cellular system. The cell-based screening protocol described in the instant invention (i.e., one which takes place inside a cell) offers many advantages over extracellular systems, because the synthesis of large quantities of RNA by enzymatic or chemical methods prior to assessing the efficacy of the siNA molecules is not necessary. The invention further describes a rapid method of using siNA libraries to identify the biological function of a gene sequence inside a cell. Applicant describes a method of using siNA libraries to identify a nucleic acid molecule, such as a gene, involved in a biological process; this nucleic acid molecule may be a known molecule with a known function, or a known molecule with a previously undefined function or an entirely novel molecule. This is a rapid means for identifying, for example, genes involved in a cellular pathway, such as cell proliferation, cell migration, cell death, and others. This method of gene discovery is not only a novel approach to studying a desired biological process but also a means to identify active reagents that can modulate this cellular process in a precise manner.

Applicant describes herein, a general approach for simultaneously assaying the ability of one or more members of a siNA library to modulate certain attributes/process(es) in a biological system, such as plants and animals or insects, involving introduction of the library into a desired cell and assaying for changes in a specific “attribute”, “characteristic” or “process”. The specific attributes include, for example, cell proliferation, cell survival, cell death, cell migration, angiogenesis, tumor volume, tumor metastasis, levels of a specific mRNA(s) in a cell, levels of a specific protein(s) in a cell, levels of a specific protein secreted, cell surface markers, cell morphology, cell differentiation pattern, cartilage degradation, transplantation, restenosis, viral replication, viral load, and the like. By modulating a specific biological pathway using a siNA library, it is possible to identify the gene(s) involved in that pathway, which can lead to the discovery of novel genes, or genes with novel function. This method provides a powerful tool to study gene function inside a cell. This approach also offers the potential for designing novel siNA constructs, identifying siNA accessible sites within a target, and for identifying new nucleic acid targets for RNAi mediated modulation of gene expression.

In one embodiment, the invention involves synthesizing a random siNA library (Random Library) and simultaneously testing all members of the Random Library in cells. This library has siNA constructs with random self complementary sequence. Cells with an altered attribute (such as inhibition of cell proliferation) as a result of interaction with the members of the Random Library are selected and the sequences of the siNA constructs from these cells are determined. Sequence information from the siNA (e.g. antisense strand sequence or sense strand sequence) can be used to isolate nucleic acid molecules that are likely to be involved in the pathway responsible for the desired cellular attribute using standard technology known in the art, e.g., nucleic acid amplification using techniques such as polymerase chain reaction (PCR).

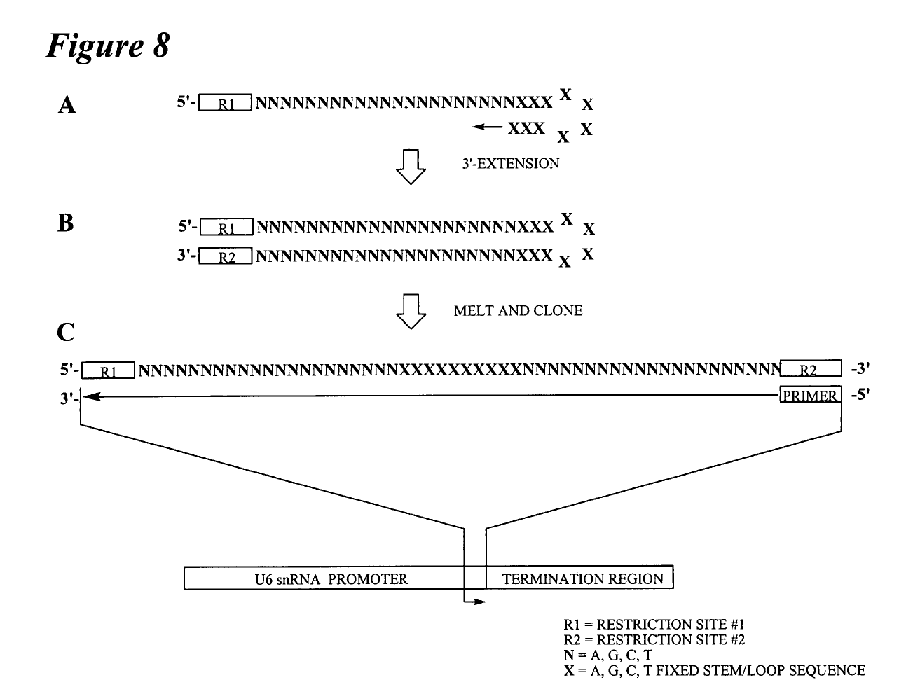

By “Random Library” as used herein is meant siNA libraries comprising all possible variants in antisense strand of the siNA. Here the complexity and the content of the library is not defined. The Random Library is expected to comprise sequences complementary to every potential target sequence, for the siNA chosen, in the genome of an organism. The Random Library can be a monomer or a multimer Random Library (see FIG. 8). By monomer Random Library is meant that a transcription unit includes one siNA unit with random self-complementary sequence. By multimer Random Library is meant that a transcription unit includes more than one siNA unit. The number of siNA units are preferably 2, 3, 4, 5, 6, 7, 8, 9, or 10. This Random Library can be used to screen for RNAi cleavage sites in a known target sequence or in an unknown target. In the first instance, the Random Library is introduced into the cell of choice and the expression of the known target gene is assayed. Cells with an altered expression of the target will yield the most effective siNA against the known target. In the second instance, the Random Library is introduced into the cell of choice and the cells are assayed for a specific attribute, for example, survival of cells. Cells that survive the interaction with the Random Library are isolated and the siNA sequence from these cells is determined. The sequence of the antisense strand of the siNA can then be used as probes to isolate the gene(s) involved in cell death. Because, the siNA(s) from the Random Library is able to modulate (e.g., down regulate) the expression of the gene(s) involved in cell death, the cells are able to survive under conditions where they would have otherwise died. This is a novel method of gene discovery. This approach not only provides the information about mediators of certain cellular processes, but also provides a means to modulate the expression of these modulators. This method can be used to identify modulators of any cell process in any organism, including but not limited to mammals, plants, and insects.

The invention provides a method for producing a class of siNA molecules which exhibit a high degree of specificity for the nucleic acid sequence of a desired target. The siNA molecule is preferably targeted to a highly conserved sequence region of a target such that specific diagnosis and/or treatment of a disease or condition can be provided with a single siNA.

In one embodiment, a method for identifying a nucleic acid molecule involved in a process in a cell is described, including the steps of: (a) synthesizing a library of siNA molecules, having a sense region and antisense region, where the antisense region and sense region have a random self complementary sequence; (b) introducing the library of siNA molecules into a cell; (c) testing the library in the cell under conditions suitable to cause the process in the cell to be altered (for example, inhibition of cell proliferation, inhibition of angiogenesis, modulation of growth and/or differentiation, and others); (d) isolating and enriching the cell with the altered process; (e) identifying and isolating the siNA in the altered cell; (f) using an oligonucleotide, having the sequence homologous to the sequence of the antisense region of the siNA isolated from the altered cell, as a probe to isolate the nucleic acid molecule from the cell or the altered cell (or optionally using genomic database data to identify the nucleic acid sequence). Those nucleic acid molecules identified using the selection/screening method described above are likely involved in the process that was being assayed for alteration by the member(s) of the siNA library. These nucleic acid molecules can be new gene sequences, or known gene sequences, with a novel function. One of the advantages of this method is that nucleic acid sequences, such as genes, involved in a biological process, such as differentiation, cell growth, disease processes including cancer, tumor angiogenesis, arthritis, cardiovascular disease, inflammation, restenosis, vascular disease and the like, can be readily identified using the Random Library approach. Thus, one Random Library for a given siNA motif can be used to assay any process in any biological system.

In another embodiment, the invention involves synthesizing a Defined siNA Library (Defined Library) and simultaneously testing it against known targets in a cell. The library includes siNA molecules having random self complementary sequence of known complexity (Defined). Modulation of expression of the target gene by siNA moelcules in the library will cause the cells to have an altered phenotype. Such cells are isolated and the siNA moelcules in these cells are the ones most suited for modulating the expression of the desired gene in the cell.

By “Defined Library” as used herein is meant a library of siNA moelcules, wherein each member siNA is designed and produced independently, then added to the library. Thus, the content, complexity (number of different siNA moelcules contained in the library) and ratios of library members are defined at the outset. A Defined Library comprises greater than 2 siNA molecules. The process involves screening the sequence of the known target RNA for all possible sites that can be cleaved by a given siNA, then synthesizing a representative number of different siNA moelcules against the target sequence, combining the siNA molecules and introducing the pooled siNA moelcules into a biological system comprising the target RNA under conditions suitable to facilitate modulation of the expression of the target RNA in said biological system.

Alternatively, certain siNA molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45. Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856.

In another aspect of the invention, RNA molecules of the present invention can be expressed from transcription units (see for example Couture et al., 1996, TIG., 12, 510) inserted into DNA or RNA vectors. The recombinant vectors can be DNA plasmids or viral vectors. siNA expressing viral vectors can be constructed based on, but not limited to, adeno-associated virus, retrovirus, adenovirus, or alphavirus. In another embodiment, pol III based constructs are used to express nucleic acid molecules of the invention (see for example Thompson, U.S. Pat. Nos. 5,902,880 and 6,146,886). The recombinant vectors capable of expressing the siNA molecules can be delivered as described above, and persist in target cells. Alternatively, viral vectors can be used that provide for transient expression of nucleic acid molecules. Such vectors can be repeatedly administered as necessary. Once expressed, the siNA molecule interacts with the target mRNA and generates an RNAi response. Delivery of siNA molecule expressing vectors can be systemic, such as by intravenous or intra-muscular administration, by administration to target cells ex-planted from a subject followed by reintroduction into the subject, or by any other means that would allow for introduction into the desired target cell (for a review see Couture et al., 1996, TIG., 12, 510).

In one aspect the invention features an expression vector comprising a nucleic acid sequence encoding at least one siNA molecule of the instant invention. The expression vector can encode one or both strands of a siNA duplex, or a single self complementary strand that self hybridizes into a siNA duplex. The nucleic acid sequences encoding the siNA molecules of the instant invention can be operably linked in a manner that allows expression of the siNA molecule (see for example Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine, advance online publication doi: 10.1038/nm725).

In another aspect, the invention features an expression vector comprising: a) a transcription initiation region (e.g., eukaryotic pol I, II or III initiation region); b) a transcription termination region (e.g., eukaryotic pol I, II or III termination region); and c) a nucleic acid sequence encoding at least one of the siNA molecules of the instant invention, wherein said sequence is operably linked to said initiation region and said termination region, in a manner that allows expression and/or delivery of the siNA molecule. The vector can optionally include an open reading frame (ORF) for a protein operably linked on the 5′ side or the 3′-side of the sequence encoding the siNA of the invention; and/or an intron (intervening sequences).

Transcription of the siNA molecule sequences can be driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as siNA in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736. The above siNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect, the invention features an expression vector comprising a nucleic acid sequence encoding at least one of the siNA molecules of the invention, in a manner that allows expression of that siNA molecule. The expression vector comprises in one embodiment; a) a transcription initiation region; b) a transcription termination region; and c) a nucleic acid sequence encoding at least one strand of the siNA molecule, wherein the sequence is operably linked to the initiation region and the termination region in a manner that allows expression and/or delivery of the siNA molecule.

In another embodiment, the expression vector comprises: a) a transcription initiation region; b) a transcription termination region; c) an open reading frame; and d) a nucleic acid sequence encoding at least one strand of a siNA molecule, wherein the sequence is operably linked to the 3′-end of the open reading frame and wherein the sequence is operably linked to the initiation region, the open reading frame and the termination region in a manner that allows expression and/or delivery of the siNA molecule. In yet another embodiment, the expression vector comprises: a) a transcription initiation region; b) a transcription termination region; c) an intron; and d) a nucleic acid sequence encoding at least one siNA molecule, wherein the sequence is operably linked to the initiation region, the intron and the termination region in a manner which allows expression and/or delivery of the nucleic acid molecule.

In another embodiment, the expression vector comprises: a) a transcription initiation region; b) a transcription termination region; c) an intron; d) an open reading frame; and e) a nucleic acid sequence encoding at least one strand of a siNA molecule, wherein the sequence is operably linked to the 3′-end of the open reading frame and wherein the sequence is operably linked to the initiation region, the intron, the open reading frame and the termination region in a manner which allows expression and/or delivery of the siNA molecule.

### EXAMPLES

The following are non-limiting examples showing the selection, isolation, synthesis and activity of nucleic acids of the instant invention.

### Example 1

### Target Discovery in Mammalian Cells

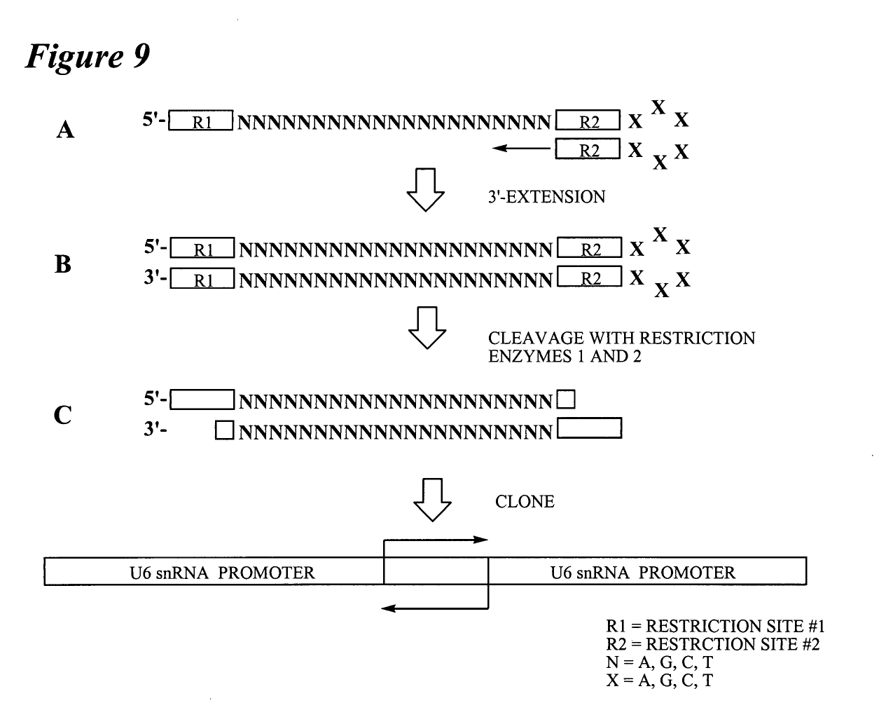

In a non-limiting example, compositions and methods of the invention are used to discover genes involved in a process of interest within mammalian cells, such as cell growth, proliferation, apoptosis, morphology, angiogenesis, differentiation, migration, viral multiplication, drug resistance, signal transduction, cell cycle regulation, or temperature sensitivity or other process. First, a randomized siNA library is generated using a process outlined in FIG. 8 for hairpin siNA constructs or in FIG. 9 for double stranded siNA constructs. These constructs are inserted into a vector capable of expressing a siNA from the library inside mammalian cells.

Reporter System

In order to discover genes playing a role in the expression of certain proteins, such as proteins involved in a cellular process described herein, a readily assayable reporter system is constructed in which a reporter molecule is co-expressed when a particular protein of interest is expressed. The reporter system consists of a plasmid construct bearing a gene coding for a reporter gene, such as Green Fluorescent Protein (GFP) or other reporter proteins known and readily available in the art. The promoter region of the GFP gene is replaced by a portion of a promoter for the protein of interest sufficient to direct efficient transcription of the GFP gene. The plasmid can also contain a drug resistance gene, such as neomycin resistance, in order to select cells containing the plasmid.

Host Cell Lines for Target Discovery

A cell line is selected as host for target discovery. The cell line is preferably known to express the protein of interest, such that upstream genes controlling the expression of the protein can be identified when modulated by a siNA construct expressed therein. The cells preferably retain protein expression characteristics in culture. The reporter plasmid is transfected into cells, for example, using a cationic lipid formulation. Following transfection, the cells are subjected to limiting dilution cloning, for example, under selection by 600 μg/mL Geneticin. Cells retaining the plasmid survive the Geneticin treatment and form colonies derived from single surviving cells. The resulting clonal cell lines are screened by flow cytometry for the capacity to upregulate GFP production. Treating the cells with, for example, sterilized M9 bacterial medium in which Pseudomonas aeruginosa had been cultured (Pseudomonas conditioned medium, PCM) is used to induce the promoter. The PCM is supplemented with phorbol myristate acetate (PMA). A clonal cell line highly responsive to promoter induction is selected as the reporter line for subsequent studies.

siNA Library Construction

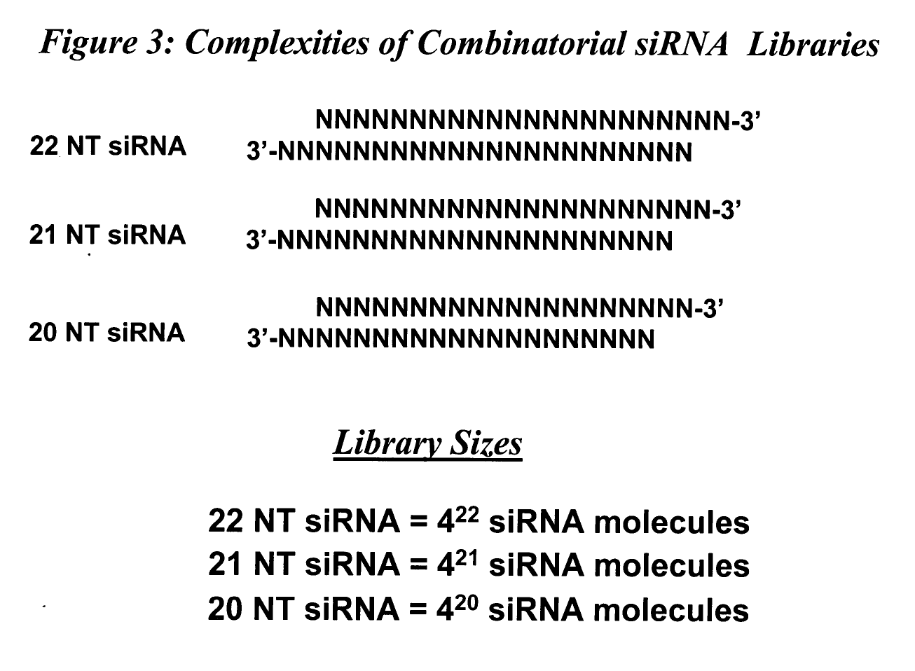

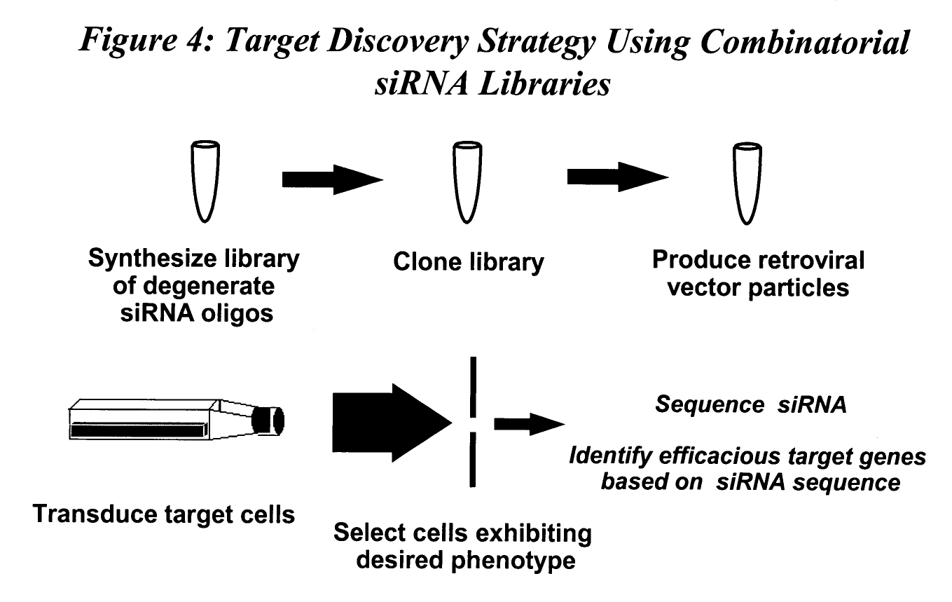

A siNA library was constructed with oligonucletides containing hairpin siNA constructs having randomized antisense regions and self complementary sense regions (see for example FIG. 8, alternately, a library of double stranded siNA constructs can be utilized as shown in FIG. 9). Oligo sequence 5′ and 3′ of the siNA contains restriction endonuclease cleavage sites for cloning. The 3′ trailing sequence forms a stem-loop for priming DNA polymerase extension to form a hairpin structure. The hairpin DNA construct is melted at 90° C. allowing DNA polymerase to generate a dsDNA construct. The double-stranded siNA library is cloned into, for example, a U6+27 transcription unit located in the 5′ LTR region of a retroviral vector containing the human nerve growth factor receptor (hNGFr) reporter gene. Positioning the U6+27/siNA transcription unit in the 5′ LTR results in a duplication of the transcription unit when the vector integrates into the host cell genome. As a result, the siNA is transcribed by RNA polyrnerase III from U6+27 and by RNA polymerase II activity directed by the 5′ LTR. The siNA library is packaged into retroviral particles that are used to infect and transduce clonal cells selected above. Assays of the hNGFr reporter are used to indicate the percentage of cells that incorporated the siNA construct. FIGS. 3 and 4 describe the generalized scheme used in the siNA library construction and target discovery. By randomized region is meant a region of completely random sequence and/or partially random sequence. By completely random sequence is meant a sequence wherein theoretically there is equal representation of A, T, G and C nucleotides or modified derivatives thereof, at each position in the sequence. By partially random sequence is meant a sequence wherein there is an unequal representation of A, T, G and C nucleotides or modified derivatives thereof, at each position in the sequence. A partially random sequence can therefore have one or more positions of complete randomness and one or more positions with defined nucleotides.

Enriching for Non-Responders to Induction

Sorting of siNA library-containing cells is performed to enrich for cells that produce less reporter GFP after treatment with the promoter inducers PCM and PMA. Lower GFP production cancan be due to RNAi activity against genes involved in the activation of the mucin promoter. Alternatively, siNA can directly target the mucin/GFP transcript resulting in reduced GFP expression.

Cells are seeded at a certain density, such as 1×10⁶ per 150 cm² style cell culture flasks and grown in the appropriate cell culture medium with fetal bovine serum. After 72 hours, the cell culture medium is replaced with serum-free medium. After 24 hours of serum deprivation, the cells are treated with serum-containing medium supplemented with PCM (to 40%) and PMA (to 50 nM) to induced GFP production. After 20 to 22 hours, cells are monitored for GFP level on, for example, a FACStar Plus cell sorter. Sorting is performed if ≧90% of siNA library cells from an unsorted control sample were induced to produce GFP above background levels. Two cell fractions are collected in each round of sorting. Following the appropriate round of sorting, the M1 fraction is selected to generate a database of siNA molecules present in the sorted cells.

Recovery of siNA Sequence from Sorted Cells

Genomic DNA is obtained from sorted siNA library cells by standard methods. Nested polymerase chain reaction (PCR) primers that hybridized to the retroviral vector 5′ and 3′ of the siNA are used to recover and amplify the siNA sequences from the particular clone of library cell DNA. The PCR product is ligated into a bacterial cloning vector. The recovered siNA library in plasmid form can be used to generate a database of siNA sequences. For example, the library is cloned into E. coli. DNA is prepared by plasmid isolation from bacterial colonies or by direct colony PCR and siNA sequence is determined. A second method can use the siNA library to transfect cloned cells. Clonal lines of stably transfected cells are established and induced with, for example, PCM and PMA. Those lines which fail to respond to GFP induction are probed by PCR for single siNA integration events. The unique siNA sequences obtained by both methods are added to a Target Sequence Tag (TST) database.

Bioinformatics

The antisense region sequences of the isolated siNA constructs are compared to public and private gene data banks. Gene matches are compiled according to perfect and imperfect matches. Potential gene targets are categorized by the number of different siNA sequences matching each gene. Genes with more than one perfect siNA match are selected for Target Validation studies.

Validation of the Target Gene

To validate a target as a regulator of protein expression, siNA reagents are designed to the target gene cDNA sequence from Genbank. The siNA reagents are complexed with a cationic lipid formulation prior to administration to cloned cells at appropriate concentrations (e.g. 5-50 nM or less). Cells are treated with siNA reagents, for example from 72 to 96 hours. Before the termination of siNA treatment, PCM (to 40%) and PMA (to 50 nM), for example, are added to induce the promoter. After twenty hours of induction the cells are harvested and assayed for phenotypic and molecular parameters. Reduced GFP expression in siNA treated cells (measured by flow cytometry) is taken as evidence for validation of the target gene. Knockdown of target RNA in siNA treated cells can correlate with reduced endogenous RNA and reduced GFP RNA to complete validation of the target.

### Example 2

### Target Discovery for Genes Associated with Mucin Production

A target discovery and target validation approach is used to find genes that are involved in chronic mucous hypersecretion involved in chronic obstructive pulmonary disease (COPD).

Reporter System

In order to discover genes playing a role in the expression of mucins, a readily assayable reporter system is devised. The reporter system consists of a plasmid construct, termed pMUC5AC-EGFP, bearing a gene coding for Green Fluorescent Protein (GFP). The promoter region of the GFP gene is replaced by a portion of the Mucin 5AC promoter sufficient to direct efficient transcription of the GFP gene. The plasmid also contains the neomycin drug resistance gene.

Host Cell Line for Target Discovery

The cell line selected as host for these studies, NCI-H292 (ATCC CRL-1848), is derived from a human lung mucoepidermoid carcinoma. The cells retain mucoepidermoid characteristics in culture and endogenously express mucin 5AC and mucin 2. The pMUC5AC-EGFP plasmid is transfected into NCI-H292 using a cationic lipid formulation. Following transfection, the cells are subjected to limiting dilution cloning under selection by 600 μg/mL Geneticin. Cells retaining the pMUC5AC-EGFP plasmid survive the Geneticin treatment and form colonies derived from single surviving cells. The resulting clonal cell lines are screened by flow cytometry for the capacity to upregulate GFP production directed by the Mucin 5AC promoter. Treating the cells with sterilized M9 bacterial medium in which Pseudomonas aeruginosa had been cultured (Pseudomonas conditioned medium, PCM) induced the mucin promoter. The PCM is supplemented with phorbol myristate acetate (PMA). A clonal cell line highly responsive to mucin promoter induction is selected as the reporter line for subsequent studies.

siNA Library Construction

A siNA library was constructed with oligonucletides containing hairpin siNA constructs having a randomized antisense regions and self complimentary sense regions (see for example FIG. 8, alternately, a library of double stranded siNA constructs can be utilized as shown in FIG. 9). Oligo sequence 5′ and 3′ of the siNA contains restriction endonuclease cleavage sites for cloning. The 3′ trailing sequence forms a stem-loop for priming DNA polymerase extension to form a hairpin structure. The hairpin DNA construct is melted at 90° C. allowing DNA polymerase to generate a dsDNA construct. The double-stranded siNA library is cloned into the U6+27 transcription unit located in the 5′ LTR region of a retroviral vector containing the human nerve growth factor receptor (hNGFr) reporter gene. Positioning the U6+27/siNA transcription unit in the 5′ LTR results in a duplication of the transcription unit when the vector integrates into the host cell genome. As a result, the siNA is transcribed by RNA polymerase III from U6+27 and by RNA polymerase II activity directed by the 5′ LTR. The siNA library is packaged into retroviral particles that were used to infect and transduce cloned cells. Assays of the hNGFr reporter are used to indicate the percentage of cells incorporated the siNA construct.

Enriching for Non-Responders to Mucin Induction

Sorting of siNA library-containing cells was performed to enrich for cells that produce less GFP after treatment with PCM and PMA. Lower GFP production can be due to RNAi activity against genes involved in the activation of the mucin promoter. Alternatively, siNA can directly target the mucin/GFP transcript resulting in reduced GFP expression.

Cells are seeded at a certain density, such as 1×10⁶ per 150 cm² style cell culture flasks and grown in the appropriate cell culture medium with fetal bovine serum. After 72 hours, the cell culture medium is replaced with serum-free cell culture medium. After 24 hours of serum deprivation the cells are treated with serum-containing medium supplemented with PCM (to 40%) and PMA (to 50 nM) to induced GFP production via the mucin promoter. After 20 to 22 hours, cells are monitored for GFP level on, for example, a FACStar Plus cell sorter. Sorting is performed if ≧90% of siNA library cells from an unsorted control sample were induced to produce GFP above background levels. Two cell fractions are collected in each round of sorting. Following the appropriate round of sorting, the M1 fraction is selected to generate a database of siNA molecules present in the sorted cells.

Recovery of siNA Sequence from Sorted Cells

Genomic DNA is obtained from sorted siNA library cells by standard methods. Nested polymerase chain reaction (PCR) primers that hybridized to the retroviral vector 5′ and 3′ of the siNA are used to recover and amplify the siNA sequences from the particular clone of library cell DNA. The PCR product is ligated into a bacterial cloning vector. The recovered siNA library in plasmid form can be used to generate a database of siNA sequences. For example, the library is cloned into E. coli. DNA is prepared by plasmid isolation from bacterial colonies or by direct colony PCR and siNA sequence is determined. A second method can use the siNA library to transfect cloned cells. Clonal lines of stably transfected cells are established and induced with PCM and PMA. Those lines which fail to respond to GFP induction are probed by PCR for single siNA integration events. The unique siNA sequences obtained by both methods are added to a Target Sequence Tag (TST) database.

Bioinformatics

The antisense region sequences of the isolated siNA constructs are compared to public and private gene data banks. Gene matches are compiled according to perfect and imperfect matches. Potential gene targets are categorized by the number of different siNA sequences matching each gene. Genes with more than one perfect siNA match are selected for Target Validation studies.

Validation of the Target Gene

To validate a target as a regulator of MUC5AC expression, siNA reagents are designed to the target gene cDNA sequence from Genbank. The siNA reagents are complexed with a cationic lipid formulation prior to administration to cloned cells at appropriate concentrations (e.g. 5-50 nM or less). Cells are treated with siNA reagents, for example from 72 to 96 hours. Before the termination of siNA treatment, PCM (to 40%) and PMA (to 50 nM) are added to induce the MUC5AC promoter. After twenty hours of induction the cells are harvested and assayed for phenotypic and molecular parameters. Reduced GFP expression in siNA treated cells (measured by flow cytometry) is taken as evidence for validation of the target gene. Knockdown of target RNA in siNA treated cells can correlate with reduced endogenous MUC5AC RNA and reduced GFP RNA (from the MUC5AC/GFP construct) to complete validation of the target.

### Example 3

### Discovery of Genes Involved in Plant Male Sterility

When two genetically distinct plant lines are crossed with each other, a variety of beneficial attributes may be combined into one single hybrid. The use of this technique for the development of hybrid seeds allows for increased agronomic benefits. Desirable attributes for plants include fruit size, growth rate, germination, yield sizes, and disease, temperature, and insect resistance. Generally speaking, this process involves generation of inbred crop lines, breeding between these lines, followed by determination whether the hybrids are superior to the original lines. For this process to be successful however, a means of preventing self-pollination must be implemented to improve cross-pollination rates. Seed generated through self-pollination would contaminate the supply of hybrid seed. By causing male or female sterility in crops, the plants would have to rely on cross breeding to reproduce. Within the context of this application, “male sterility” is defined as a condition in which a plant has functional female reproductive organs but is incapable of self-fertilization. Fertilization of the embryo sac will occur only when the pollen of a second flower comes into contact with the female organs. Alternatively “female sterility” is defined as a condition in which a plant cannot produce viable seeds because of abnormal functioning of the female gametophyte, female gamete, female zygote, or the seed.

Some plants such as corn have spatially separated male and female organs, and therefore removal of the fertile pollen from the plant is sufficient to prevent self-pollination. While functional in corn, this strategy cannot be transferred to other major crop plants since the male and female organs are present within the same flower. Therefore removal of the fertile pollen becomes cumbersome and in may cases economically infeasible. Several strategies for preventing self pollination have been suggested which include chemical and genetic sterilization.

Chemical sterilization involves the use of compounds known as gametocides which can temporarily cause male sterility. The compounds function by killing or blocking pollen production within the flower. The cost of these compounds can be limiting especially since the gametocide must be applied with every occurrence of flower production. Any new flowers that develop following the initial spraying must also be sprayed to prevent cross pollination. The timing of gametocide spraying must be carefully implemented to coincide with flower production, which can be problematic because of the difficulty in anticipating the appearance of flowers.

Another mechanism is called cytoplasmic male sterility (CMS) in which a defective mitochondrion causes an inhibition or obstruction of pollen production. Alternatively, the prevention of pollen production can involve alterations within the cell's nucleus. The present invention decribes a process for indentification of genes involved in male and/or female sterility. Applicant believes that siNA technology offers an attractive new means to alter gene expression in plants and to discover new genes involved in male and/or female sterility.

Thus in one aspect of the invention, applicant describes a method for the identification of genes involved in male or female sterility. The Random Library approach is used to discover genes whose down-regulation results in a male sterile phenotype. These genes will likely be involved in microspore, tapetum, filament, pollen and anther formation, as well as anther dehiscence. Examples of known genes involved in male sterile phenotype include Jag18 (WO 97/30581) and gene whose peptide sequence is given in U.S. Pat. No. 5,478,369. The method described herein requires no initial sequence information but allows for sequence information to be obtained in plants demonstrating the desired phenotype.

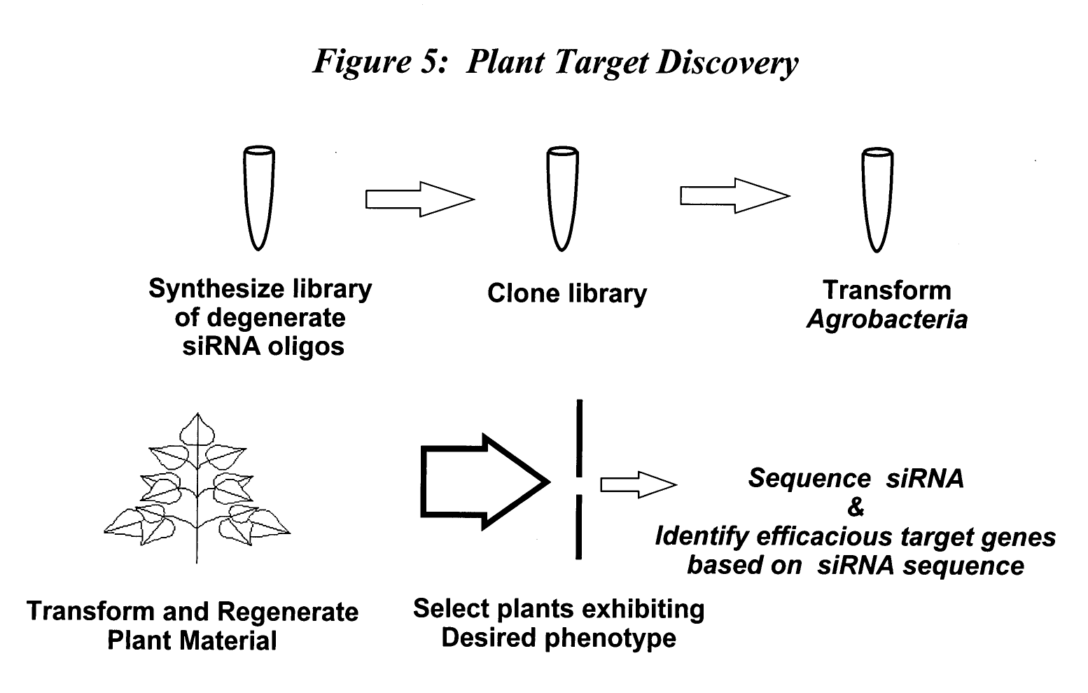

One non-limiting method for the identification of male sterility gene(s) is illustrated in FIG. 5. A Random Library is constructed from oligonucleotides containing randomized siNA molecules having complementary sense and antisense regions. The expected frequency of seeing a desired phenotype is related to the antisense region length in the library (Random Library) and the number of genes involved in the phenotype. For a siNA library, for where the siNA is designed having 21 nucleotides in each sense and antisense region, this represents 4²¹ siNA molecules. A Multimer Random Library of siNAs with an average of 10 siNA units covalently attached to each other (approximately 420 nucleotides long) is synthesized to reduce the number of clones that have to be transfected. The Random library of siNA is transcribed and cloned into expression vectors using methods described in FIGS. 8 and 9. The plasmid can also include a gene which confers resistance to a cytotoxic substance (e.g. chlorosulfuron, hygromyacin, PAT and/or bar, bromoxynil, kanamycin and the like), which allows for selection following transfection. These clones are then used to transform agrobacterium using techniques familiar to those skilled in the art (U.S. Pat. No. 5,177,010 to University of Toledo, U.S. Pat. No. 5,104,310 to Texas A&M, European Patent Application 0131624B1, European Patent Applications 120516, 159418B1 and 176,112 to Schilperoot, U.S. Pat. Nos. 5,149,645, 5,469,976, 5,464,763 and 4,940,838 and 4,693,976 to Schilperoot, European Patent Applications 116718, 290799, 320500 all to MaxPlanck, European Patent Applications 604662 and 627752 to Japan Tobacco, European Patent Applications 0267159, and 0292435 and U.S. Pat. No. 5,231,019 all to Ciba Geigy, U.S. Pat. Nos. 5,463,174 and 4,762,785 both to Calgene, and U.S. Pat. Nos. 5,004,863 and 5,159,135 both to Agracetus; all are incorporated by reference herein). The recombinant agrobacterium is then used to transform a single plant cell which is capable of regenerating into a whole plant. Other transfection technologies can also be utilized to deliver DNA plasmids into the plant cell including but not limited to electroporation, liposomes, cationic lipids, CaCl₂ precipitation and the like known in the art. The plants cells are then grown into a whole plant and analyzed to determine if complete or partial male sterility exists. Complete male sterility is defined as the state wherein no pollen is produced and/or released causing an inability of the plant to self-fertilize. Partial male sterility is defined as the state wherein reduced or abnormal pollen production or release results compared to normal wild type plants.

To allow for easier observation of sterility in Arabidopsis plant, a strain expressing Green Florescent Protein (GFP) under the control of a pollen-specific promoter is generated. The Arabidopsis line is then transformed with siNA libraries expressed under the control of different promoters. A constitutive promoter (such as the CaMV 35S) is utilized for siNA expression while a pollen or anther specific promoter is used for the expression of GFP. The constitutively expressed siNA(s) from the Random library is likely to identify genes that are tissue specifically regulated under the control of male fertility. The random library comprised of a tissue specific promoter might be able identify genes which are not directly related to reproduction but whose inhibition may nontheless cause male and/or female sterility (e.g. housekeeping genes such as actin). Any reduction in fluorescence is an indication that the inhibition of a gene is linked to or involved in male sterility.

From the plants demonstrating complete or partial male sterility, RNA is purified and the siNA RNA is amplified and cloned by RT-PCR. Alternatively or in addition, the siNA gene is directly amplified from the genomic DNA using standard molecular biology techniques known in the art. This siNA is recloned and retransformed as described above to ensure (confirm) that the phenotype was change is due to siNA activity and not due to any insertional mutagenesis. If the trait is recreated, the sequence of the siNA binding arm is used as a tag to find the gene involved in the modification of phenotype. Using bioinformatics, available sequences is searched for homology. If no related sequence is found, cDNA libraries can be screened using the 15 nucleotide binding arm sequence as a probe to isolate the gene from the plant using standard molecular biology techniques known in the art.

In yet another aspect of the invention, hybrid seed plants are produced in which one or more genes involved in male sterility are completely or partially inhibited. These genes are individually or in combination inhibited either using the siNA(s) that was responsible for the gene's indentification, or using other siNAs. The transgenic plant, where one or more of the male sterility genes is inhibited, is mated with a suitable male fertile plant causing the synthesis of hybrid seeds. Applicant has developed a method for not only identifying gene(s) involved in biochemical pathways in plants, but has in the process can develop a siNA that can then be used to specifically down-regulate that gene in plants.

### Example 4

### Selection of siNA Target Sites Used to Validate a Gene Target

The following non-limiting steps can be used to carry out the selection of siNAs targeting a given gene sequence or transcipt.
1. The target sequence is parsed in silico into a list of all fragments or subsequences of a particular length, for example 23 nucleotide fragments, contained within the target sequence. This step is typically carried out using a custom Perl script, but commercial sequence analysis programs such as Oligo, MacVector, or the GCG Wisconsin Package can be employed as well.
2. In some instances the siNAs correspond to more than one target sequence; such would be the case for example in targeting many different strains of a viral sequence, for targeting different transcipts of the same gene, targeting different transcipts of more than one gene, or for targeting both the human gene and an animal homolog. In this case, a subsequence list of siNA reagents of a particular length is generated for each of the targets, and then the lists are compared to find matching sequences in each list. The subsequences are then ranked according to the number of target sequences that contain the given subsequence; the goal is to find subsequences that are present in most or all of the target sequences. Alternately, the ranking can indentify subsequences that are unique to a target sequence, such as a mutant target sequence. Such an approach would enable the use of siNA to target specifically the mutant sequence and not affect the expression of the normal sequence.
3. In some instances the siNA subsequences are absent in one or more sequences while present in the desired target sequence; such would be the case if the siNA targets a gene with a paralogous family member that is to remain untargeted. As in case 2 above, a subsequence list of a particular length is generated for each of the targets, and then the lists are compared to find sequences that are present in the target gene but are absent in the non-targeted paralog.
4. The ranked siNA subsequences can be further analyzed and ranked according to GC content. A preference can be given to sites containing 30-70% GC, with a further preference to sites containing 40-60% GC.
5. The ranked siNA subsequences can be further analyzed and ranked according to self-folding and internal hairpins. Weaker internal folds are preferred; strong hairpin structures are to be avoided.
6. The ranked siNA subsequences can be further analyzed and ranked according to whether they have runs of GGG or CCC in the sequence. GGG (or even more Gs) in either strand can make oligonucleotide synthesis problematic, so it is avoided whenever better sequences are available. CCC is searched in the target strand because that will place GGG in the opposite strand.
7. The ranked siNA subsequences can be further analyzed and ranked according to whether they have the dinucleotide UU (uridine dinucleotide) on the 3′ end of the sequence, and/or AA on the 5′ end of the sequence (to yield 3′ UU on the antisense sequence). These sequences allow one to design siNA molecules with terminal TT thymidine dinucleotides.
8. Four to ten target sites are chosen from the ranked list of subsequences as described above. For example, in subsequences having 23 nucleotides, the right 21 nucleotides of each chosen 23-mer subsequence are then designed and synthesized for the upper (sense) strand of the siNA duplex, while the reverse complement of the left 21 nucleotides of each chosen 23-mer subsequence are then designed and synthesized for the lower (antisense) strand of the siNA duplex. If terminal TT residues are desired for the sequence (as described in case 7), then the two 3′ terminal nucleotides of both the sense and antisense strands are replaced by TT prior to synthesizing the oligos.
9. The siNA molecules are screened in an in vitro, cell culture or animal model system to identify the most active siNA molecule or the most preferred target site within the target RNA sequence.

### Example 5

### siNA Design for Target Validation

siNA target sites were chosen by analyzing sequences of the RNA target and optionally prioritizing the target sites on the basis of folding (structure of any given sequence analyzed to determine siNA accessibility to the target), or alternately by using an in vitro siNA system as described in Example 7 herein. siNA molecules are designed that could bind each target and are optionally individually analyzed by computer folding to assess whether the siNA molecule can interact with the target sequence. Varying the length of the siNA molecules can optionally be done to optimize activity. Generally, a sufficient number of complementary nucleotide bases are chosen to bind to, or otherwise interact with, the target RNA, but the degree of complementarity can be modulated to accommodate siNA duplexes of varying length or base composition. By using such methodologies, siNA molecules can be designed to target sites within any known RNA sequence, for example those RNA sequences corresponding to the any gene transcript.

### Example 6

### Chemical Synthesis and Purification of siNA

siNA molecules can be designed to interact with various sites in the RNA message, for example target sequences within the RNA sequences described herein. The sequence of one strand of the siNA molecule(s) is complementary to the target site sequences described above. The siNA molecules can be chemically synthesized using methods described herein. Inactive siNA molecules that are used as control sequences can be synthesized by scrambling the sequence of the siNA molecules such that it is not complementary to the target sequence. Generally, siNA constructs can by synthesized using solid phase oligonucleotide synthesis methods as described herein (see for example Usman et al., U.S. Pat. Nos. 5,804,683; 5,831,071; 5,998,203; 6,117,657; 6,353,098; 6,362,323; 6,437,117; 6,469,158; Scaringe et al., U.S. Pat. Nos. 6,111,086; 6,008,400; 6,111,086 all incorporated by reference herein in thier entirety).

In a non-limiting example, RNA oligonucleotides are synthesized in a stepwise fashion using the phosphoramidite chemistry as is known in the art. Standard phosphoramidite chemistry involves the use of nucleosides comprising any of 5′-O-dimethoxytrityl, 2′-O-tert-butyldimethylsilyl, 3′-O-2-Cyanoethyl N,N-diisopropylphosphoroamidite groups, and exocyclic amine protecting groups (e.g. N6-benzoyl adenosine, N4 acetyl cytidine, and N2-isobutyryl guanosine). Alternately, 2′-O-Silyl Ethers can be used in conjunction with acid-labile 2′-O-orthoester protecting groups in the synthesis of RNA as described by Scaringe supra. Differing 2′ chemistries can require different protecting groups, for example 2′-deoxy-2′-amino nucleosides can utilize N-phthaloyl protection as described by Usman et al., U.S. Pat. No. 5,631,360, incorporated by reference herein in its entirety).

During solid phase synthesis, each nucleotide is added sequentially (3′- to 5′-direction) to the solid support-bound oligonucleotide. The first nucleoside at the 3′-end of the chain is covalently attached to a solid support (e.g., controlled pore glass or polystyrene) using various linkers. The nucleotide precursor, a ribonucleoside phosphoramidite, and activator are combined resulting in the coupling of the second nucleoside phosphoramidite onto the 5′-end of the first nucleoside. The support is then washed and any unreacted 5′-hydroxyl groups are capped with a capping reagent such as acetic anhydride to yield inactive 5′-acetyl moieties. The trivalent phosphorus linkage is then oxidized to a more stable phosphate linkage. At the end of the nucleotide addition cycle, the 5′-O-protecting group is cleaved under suitable conditions (e.g., acidic conditions for trityl-based groups and Fluoride for silyl-based groups). The cycle is repeated for each subsequent nucleotide.

Modification of synthesis conditions can be used to optimize coupling efficiency, for example by using differing coupling times, differing reagent/phosphoramidite concentrations, differing contact times, differing solid supports and solid support linker chemistries depending on the particular chemical composition of the siNA to be synthesized. Deprotection and purification of the siNA can be performed as is generally described in Vargeese et al., U.S. Ser. No. 10/194,875 incorporated by reference herein in its entirety or Scaringe supra,. Additionally, deprotection conditions can be modified to provide the best possible yield and purity of siNA constructs. For example, applicant has observed that oligonucleotides comprising 2′-deoxy-2′-fluoro nucleotides can degrade under inapproprate deprotection conditions. Such oligonucleotides are deprotected using aqueous methylamine at about 35° C. for 30 minutes. If the 2′-deoxy-2′-fluoro containing oligonucleotide also comprises ribonucleotides, after deprotection with aqueous methylamine at about 35° C. for 30 minutes, TEA-HF is added and the reaction maintained at about 65° C. for an additional 15 minutes.

### Example 7

### RNAi in Vitro Assay to Assess siNA Activity

An in vitro assay that recapitulates RNAi in a cell free system can be used to evaluate siNAs targeting RNA targets. The assay comprises the system described by Tuschl et al., 1999, Genes and Development, 13, 3191-3197 and Zamore et al., 2000, Cell, 101, 25-33 adapted for use with target RNA. A Drosophila extract derived from syncytial blastoderm is used to reconstitute RNAi activity in vitro. Target RNA is generated via in vitro transcription from an appropriate plasmid using T7 RNA polymerase or via chemical synthesis as described herein. Sense and antisense siNA strands (for example 20 uM each) are annealed by incubation in buffer (such as 100 mM potassium acetate, 30 mM HEPES-KOH, pH 7.4, 2 mM magnesium acetate) for 1 min. at 90° C. followed by 1 hour at 37° C., then diluted in lysis buffer (for example 100 mM potassium acetate, 30 mM HEPES-KOH at pH 7.4, 2 mM magnesium acetate). Annealing can be monitored by gel electrophoresis on an agarose gel in TBE buffer and stained with ethidium bromide. The Drosophila lysate is prepared using zero to two hour old embryos from Oregon R flies collected on yeasted molasses agar that are dechorionated and lysed. The lysate is centrifuged and the supernatant isolated. The assay comprises a reaction mixture containing 50% lysate [vol/vol], RNA (10-50 pM final concentration), and 10% [vol/vol] lysis buffer containing siNA (10 nM final concentration). The reaction mixture also contains 10 mM creatine phosphate, 10 ug.ml creatine phosphokinase, 100 um GTP, 100 uM UTP, 100 uM CTP, 500 uM ATP, 5 mM DTT, 0.1 U/uL RNasin (Promega), and 100 uM of each amino acid. The final concentration of potassium acetate is adjusted to 100 mM. The reactions are pre-assembled on ice and preincubated at 25° C. for 10 minutes before adding RNA, then incubated at 25° C. for an additional 60 minutes. Reactions are quenched with 4 volumes of 1.25× Passive Lysis Buffer (Promega). Target RNA cleavage is assayed by RT-PCR analysis or other methods known in the art and are compared to control reactions in which siNA is omitted from the reaction.

Alternately, internally-labeled target RNA for the assay is prepared by in vitro transcription in the presence of [a-³²P] CTP, passed over a G 50 Sephadex column by spin chromatography and used as target RNA without further purification. Optionally, target RNA is 5′-³²P-end labeled using T4 polynucleotide kinase enzyme. Assays are performed as described above and target RNA and the specific RNA cleavage products generated by RNAi are visualized on an autoradiograph of a gel. The percentage of cleavage is determined by Phosphor Imager® quantification of bands representing intact control RNA or RNA from control reactions without siNA and the cleavage products generated by the assay.

In one embodiment, this assay is used to determine target sites within the RNA target for siNA mediated RNAi cleavage, wherein a plurality of siNA constructs are screened for RNAi mediated cleavage of the RNA target, for example by analysing the assay reaction by electrophoresis of labelled target RNA, or by northern blotting, as well as by other methodology well known in the art.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations that are not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

1. A method for identifying a nucleic acid molecule capable of modulating a process in a biological system comprising the steps of:
a) introducing a library of siNA constructs into a biological system under conditions suitable for modulating a process therein; and
b) determining the nucleotide sequence of at least a portion of a siNA construct from the biological system in which a process has been modulated to identify the nucleic acid molecule capable of modulating a process in the biological system.

2. A method for identifying one or more nucleic acid molecules involved in a process in a biological system comprising the steps of:
a) introducing a library of siNA constructs into a biological system under conditions suitable for modulating a process therein;
b) identifying a siNA construct(s) present in the biological system in which a process has been altered; and
c) determining the nucleotide sequence of at least a portion of a siNA construct from (b) to identify one or more nucleic acid molecule(s) involved in a process in the biological system.

3. A method for identifying a siNA construct capable of modulating a process in a biological system comprising the steps of:
a) introducing a library of siNA constructs into a biological system under conditions suitable for modulating a process therein; and
b) identifying a siNA construct from the biological system in which a process has been modulated.

4. The method of claim 1, wherein said biological system is of mammalian origin.

5. The method of claim 2, wherein said biological system is of mammalian origin.

6. The method of claim 3, wherein said biological system is of mammalian origin.

7. The method of claim 4, wherein said biological system is of human origin.

8. The method of claim 5, wherein said biological system is of human origin.

9. The method of claim 6, wherein said biological system is of human origin.

10. The method of claim 1, wherein said siNA is a double stranded RNA having self complementary sense and antisense regions.

11. The method of claim 2, wherein said siNA is a double stranded RNA having self complementary sense and antisense regions.

12. The method of claim 3, wherein said siNA is a double stranded RNA having self complementary sense and antisense regions.

13. The method of claim 1, wherein said siNA is a single stranded RNA.

14. The method of claim 2, wherein said siNA is a single stranded RNA.

15. The method of claim 3, wherein said siNA is a single stranded RNA.

16. The method of claim 13, wherein said single stranded siNA has self complementary sense and antisense regions.

17. The method of claim 14, wherein said single stranded siNA has self complementary sense and antisense regions.

18. The method of claim 15, wherein said single stranded siNA has self complementary sense and antisense regions.

19. The method of claim 1, wherein said process is selected from the group consisting of growth, proliferation, apoptosis, morphology, angiogenesis, differentiation, migration, viral multiplication, drug resistance, signal transduction, cell cycle regulation, temperature sensitivity and chemical sensitivity.

20. The method of claim 2, wherein said process is selected from the group consisting of growth, proliferation, apoptosis, morphology, angiogenesis, differentiation, migration, viral multiplication, drug resistance, signal transduction, cell cycle regulation, temperature sensitivity and chemical sensitivity.

21. The method of claim 3, wherein said process is selected from the group consisting of growth, proliferation, apoptosis, morphology, angiogenesis, differentiation, migration, viral multiplication, drug resistance, signal transduction, cell cycle regulation, temperature sensitivity and chemical sensitivity.

22. The method of claim 1, wherein said library of siNA constructs comprises siNA constructs encoded by an expression vector in a manner which allows expression of said nucleic acid siNA constructs.

23. The method of claim 2, wherein said library of siNA constructs comprises siNA constructs encoded by an expression vector in a manner which allows expression of said nucleic acid siNA constructs.

24. The method of claim 3, wherein said library of siNA constructs comprises siNA constructs encoded by an expression vector in a manner which allows expression of said nucleic acid siNA constructs.

25. The method of claim 22, wherein said expression vector comprises:
a) a transcription initiation region;
b) a transcription termination region; and
c) a gene encoding at least one siNA,
wherein the gene is operably linked to the initiation region and the termination region in a manner which allows expression or delivery of the siNA or both.

26. The method of claim 23, wherein said expression vector comprises:
a) a transcription initiation region;
b) a transcription termination region; and
c) a gene encoding at least one siNA,
wherein the gene is operably linked to the initiation region and the termination region in a manner which allows expression or delivery of the siNA or both.

27. The method of claim 24, wherein said expression vector comprises:
a) a transcription initiation region;
b) a transcription termination region; and
c) a gene encoding at least one siNA,
wherein the gene is operably linked to the initiation region and the termination region in a manner which allows expression or delivery of the siNA or both.

28. The method of claim 25, wherein the expression vector comprises:
a) a transcription initiation region;
b) a transcription termination region;
c) an open reading frame; and
d) a gene encoding at least one siNA,
wherein the gene is operably linked to the 3′-end of the open reading frame and wherein the gene is operably linked to the initiation region, the open reading frame and the termination region in a manner which allows expression or delivery of the siNA, or both.

29. The method of claim 26, wherein the expression vector comprises:
a) a transcription initiation region;
b) a transcription termination region;
c) an open reading frame; and
d) a gene encoding at least one siNA,
wherein the gene is operably linked to the 3′-end of the open reading frame and wherein the gene is operably linked to the initiation region, the open reading frame and the termination region in a manner which allows expression or delivery of the siNA, or both.

30. The method of claim 27, wherein the expression vector comprises:
a) a transcription initiation region;
b) a transcription termination region;
c) an open reading frame; and
d) a gene encoding at least one siNA,
wherein the gene is operably linked to the 3′-end of the open reading frame and wherein the gene is operably linked to the initiation region, the open reading frame and the termination region in a manner which allows expression or delivery of the siNA, or both.

31. The method of claim 25, wherein the expression vector comprises:
a) a transcription initiation region;
b) a transcription termination region;
c) an intron; and
d) a gene encoding at least one siNA,
wherein the gene is operably linked to the initiation region, the intron and the termination region in a manner which allows expression or delivery of the siNA or both.

32. The method of claim 26, wherein the expression vector comprises:
a) a transcription initiation region;
b) a transcription termination region;
c) an intron; and
d) a gene encoding at least one siNA,
wherein the gene is operably linked to the initiation region, the intron and the termination region in a manner which allows expression or delivery of the siNA or both.

33. The method of claim 27, wherein the expression vector comprises:
a) a transcription initiation region;
b) a transcription termination region;
c) an intron; and
d) a gene encoding at least one siNA,
wherein the gene is operably linked to the initiation region, the intron and the termination region in a manner which allows expression or delivery of the siNA or both.

34. The method of claim 25, wherein the expression vector comprises:
a) a transcription initiation region;
b) a transcription termination region;
c) an intron;
d) an open reading frame; and
e) a gene encoding at least one siNA,
wherein the gene is operably linked to the 3′-end of the open reading frame and wherein the gene is operably linked to the initiation region, the intron, the open reading frame and the termination region in a manner which allows expression or delivery of the siNA or both.

35. The method of claim 26, wherein the expression vector comprises:
a) a transcription initiation region;
b) a transcription termination region;
c) an intron;
d) an open reading frame; and
e) a gene encoding at least one siNA,
wherein the gene is operably linked to the 3′-end of the open reading frame and wherein the gene is operably linked to the initiation region, the intron, the open reading frame and the termination region in a manner which allows expression or delivery of the siNA or both.

36. The method of claim 27, wherein the expression vector comprises:
a) a transcription initiation region;
b) a transcription termination region;
c) an intron;
d) an open reading frame; and
e) a gene encoding at least one siNA,
wherein the gene is operably linked to the 3′-end of the open reading frame and wherein the gene is operably linked to the initiation region, the intron, the open reading frame and the termination region in a manner which allows expression or delivery of the siNA or both.

37. The method of claim 25, wherein the expression vector is derived from a retrovirus.

38. The method of claim 26, wherein the expression vector is derived from a retrovirus.

39. The method of claim 27, wherein the expression vector is derived from a retrovirus.

40. The method of claim 25, wherein the expression vector is derived from an adenovirus.

41. The method of claim 26, wherein the expression vector is derived from an adenovirus.

42. The method of claim 27, wherein the expression vector is derived from an adenovirus.

43. The method of claim 25, wherein the expression vector is derived from an adeno-associated virus.

44. The method of claim 26, wherein the expression vector is derived from an adeno-associated virus.

45. The method of claim 27, wherein the expression vector is derived from an adeno-associated virus.

46. The method of claim 25, wherein the expression vector is derived from an alphavirus.

47. The method of claim 26, wherein the expression vector is derived from an alphavirus.

48. The method of claim 27, wherein the expression vector is derived from an alphavirus.

49. The method of claim 25, wherein the expression vector is derived from a bacterial plasmid.

50. The method of claim 26, wherein the expression vector is derived from a bacterial plasmid.

51. The method of claim 27, wherein the expression vector is derived from a bacterial plasmid.

52. The method of claim 25, wherein the expression vector is operably linked to a RNA polymerase II promoter element.

53. The method of claim 26, wherein the expression vector is operably linked to a RNA polymerase II promoter element.

54. The method of claim 27, wherein the expression vector is operably linked to a RNA polymerase II promoter element.

55. The method of claim 25, wherein the expression vector is operably linked to a RNA polymerase III promoter element.

56. The method of claim 26, wherein the expression vector is operably linked to a RNA polymerase III promoter element.

57. The method of claim 27, wherein the expression vector is operably linked to a RNA polymerase III promoter element.

58. The method of claim 55, wherein the RNA polymerase III promoter is derived from a transfer RNA gene.

59. The method of claim 56, wherein the RNA polymerase III promoter is derived from a transfer RNA gene.

60. The method of claim 57, wherein the RNA polymerase III promoter is derived from a transfer RNA gene.

61. The method of claim 55, wherein the RNA polymerase III promoter is derived from a U6 small nuclear RNA gene.

62. The method of claim 56, wherein the RNA polymerase III promoter is derived from a U6 small nuclear RNA gene.

63. The method of claim 57, wherein the RNA polymerase III promoter is derived from a U6 small nuclear RNA gene.

64. The method of claim 55, wherein the siNA transcript comprises a sequence at its 5′-end homologous to the terminal 27 nucleotides encoded by the U6 small nuclear RNA gene.

65. The method of claim 56, wherein the siNA transcript comprises a sequence at its 5′-end homologous to the terminal 27 nucleotides encoded by the U6 small nuclear RNA gene.

66. The method of claim 57, wherein the siNA transcript comprises a sequence at its 5′-end homologous to the terminal 27 nucleotides encoded by the U6 small nuclear RNA gene.

67. The method of claim 64, wherein the RNA polymerase III promoter is derived from a TRZ RNA gene.

68. The method of claim 65, wherein the RNA polymerase III promoter is derived from a TRZ RNA gene.

69. The method of claim 66, wherein the RNA polymerase III promoter is derived from a TRZ RNA gene.

70. The method of claim 1, wherein the biological system is of a eukaryotic origin.

71. The method of claim 2, wherein the biological system is of a eukaryotic origin.

72. The method of claim 3, wherein the biological system is of a eukaryotic origin.

73. The method of claim 1, wherein the siNA is of length sufficient to mediate RNAi.

74. The method of claim 2, wherein the siNA is of length sufficient to mediate RNAi.

75. The method of claim 3, wherein the siNA is of length sufficient to mediate RNAi.

76. The method of claim 73, wherein the siNA comprises a sense and antisense region, each having a length of about 18 to about 23 nucleotides.

77. The method of claim 74, wherein the siNA comprises a sense and antisense region, each having a length of about 18 to about 23 nucleotides.

78. The method of claim 75, wherein the siNA comprises a sense and antisense region, each having a length of about 18 to about 23 nucleotides.

79. The method of claim 73, wherein the siNA comprises a 3′-nucleotide overhang of about 1 to about 3 nucleotides in the sense region, of the antisense region, or both the sense and antisense regions of the siNA.

80. The method of claim 74, wherein the siNA comprises a 3′-nucleotide overhang of about 1 to about 3 nucleotides in the sense region, of the antisense region, or both the sense and antisense regions of the siNA.

81. The method of claim 75, wherein the siNA comprises a 3′-nucleotide overhang of about 1 to about 3 nucleotides in the sense region, of the antisense region, or both the sense and antisense regions of the siNA.

82. The method of claim 79, wherein the 3′-nucleotide overhang comprises 2 nucleotides.

83. The method of claim 80, wherein the 3′-nucleotide overhang comprises 2 nucleotides.

84. The method of claim 81, wherein the 3′-nucleotide overhang comprises 2 nucleotides.

85. The method of claim 1, wherein the library of siNA constructs is a random library.

86. The method of claim 2, wherein the library of siNA constructs is a random library.

87. The method of claim 3, wherein the library of siNA constructs is a random library.

88. The method of claim 85, wherein the random library of siNA constructs is a multimer random library.

89. The method of claim 86, wherein the random library of siNA constructs is a multimer random library.

90. The method of claim 87, wherein the random library of siNA constructs is a multimer random library.

91. The method of claim 88, wherein the multimer random library comprises at least one siNA.

92. The method of claim 89, wherein the multimer random library comprises at least one siNA.

93. The method of claim 90, wherein the multimer random library comprises at least one siNA.

94. A method for identifying a family of siNA constructs capable of modulating a process in a biological system comprising the steps of:
a) introducing a library of siNA constructs into a biological system under conditions suitable for modulating a process therein; and
b) identifying a family of siNA constructs from the biological system in which a process has been modulated.

95. A method for identifying a family of nucleic acid molecules capable of modulating a process in a biological system comprising the steps of:
a) introducing a library of siNA constructs into a biological system under conditions suitable for modulating a process therein; and
(b) identifying a family of siNA constructs present in the biological system in which a process has been altered; and
(c) determining the nucleotide sequence of at least a portion of the family of siNA constructs identified in (b) to identify the family of nucleic acid molecules involved in a process in the biological system.

96. The method of claim 1, wherein the siNA is chemically modified.

97. The method of claim 2, wherein the siNA is chemically modified.

98. The method of claim 3, wherein the siNA is chemically modified.
